SEQUENCE LISTING (6 sequences)

SEQ ID NO: 1 - DNA, 26 bp, Artificial sequence
  Synthetic primer
catgaacaagagatgattaggcagag

SEQ ID NO: 2 - DNA, 22 bp, Artificial sequence
  Synthetic primer
ggaggctgtaggcataaattgg

SEQ ID NO: 3 - DNA, 20 bp, Artificial sequence
  Synthetic probe
ctgcgcaccagcaccatgca

SEQ ID NO: 4 - DNA, 19 bp, Artificial sequence
  Synthetic primer
gcagatgagaaggcacaga

SEQ ID NO: 5 - DNA, 17 bp, Artificial sequence
  Synthetic primer
cttctccgtctgccgtt

SEQ ID NO: 6 - DNA, 22 bp, Artificial sequence
  Synthetic probe
agtccgcgtaaagagaggtgcg

(19) United States
(12) Patent Application Publication
(10) Pub. No.: US 2019/0070165 A1
(43) Pub. Date: Mar. 7, 2019

(54) N-HYDROXYISOQUINOLINEDIONE INHIBITORS OF HBV REPLICATION

(71) Applicants: Saint Louis University, St. Louis, MO (US); Ecole Nationale Supérieure de Chimie de Lille, Villeneuve-D'Ascq (FR)

(72) Inventors: John Edwin TAVIS, Kirkwood, MT (US); Philippe COTELLE, Lille (FR); Fabrice BAILLY, Lille (FR)

(73) Assignees: Saint Louis University, St. Louis, MO (US); Ecole Nationale Supérieure de Chimie de Lille, Villeneuve-D'Ascq (FR)

(21) Appl. No.: 16/085,241

(22) PCT Filed: Mar. 16, 2017
(86) PCT No.: PCT/US2017/022738

§ 371 Date: Sep. 14, 2018

Related U.S. Application Data
(60) Provisional application No. 62/309,332, filed on Mar. 16, 2016.

(51) Int. Cl. A61K 31/472; A61P 31/14

(52) U.S. Cl. CPC A61K 31/472; A61K 31/513; A61P 31/14

(57) ABSTRACT

In some aspects, the present disclosure provides compounds of the formula: Formula (I) wherein the variables are defined herein are provided, which may be used to inhibit viral replication. In some embodiments, these compounds may be used to treat an infection of hepatitis B virus.

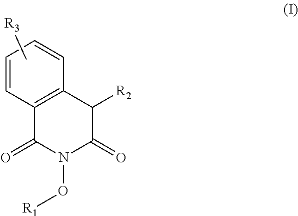

The present application claims benefit of priority to U.S. Provisional Application Ser. No. 62/309,332, filed Mar. 16, 2016, the entire contents of which are hereby incorporated by reference.

The invention was made with government support under Grants No. R01 AI104494, U01 DK082871, and R03 AI109460 awarded by the National Institutes of Health and UL1 TR000448 awarded by the National Institutes of Health and the National Center for Advancing Translational Sciences. The government has certain rights in the invention.

### BACKGROUND

### I. Field

The disclosure relates to the fields of pathology, virology, molecular biology and pharmaceuticals. More specifically, the disclosure relates to inhibitors for the treatment and prevention of hepatitis B diseases.

### II. Related Art

Hepatitis B virus (HBV) is a hepatotropic DNA virus that replicates by reverse transcription (Hostomsky et al., 1993). It chronically infects >350 million people world-wide and kills up to 1.2 million patients annually by inducing liver failure and liver cancer (Steitz, 1995; Katayanagi et al., 1990; Yang et al., 1990; Lai et al., 2000). Reverse transcription is catalyzed by a virally-encoded polymerase that has two enzymatic activities: a DNA polymerase that synthesizes new DNA and a ribonuclease H (RNAseH) that destroys the viral RNA after it has been copied into DNA (Hostomsky et al., 1993; Rice et al., 2001; Hickman et al., 1994; Ariyoshi et al., 1994). Both activities are essential for viral replication.

HBV infections are treated with interferon α or one of five nucleos(t)ide analogs (Parker et al., 2004; Song et al., 2004; Lima et al., 2001). Interferon α leads to sustained clinical improvement in 20-30% of patients, but the infection is very rarely cleared (Hostomsky et al., 1993; Katayanagi et al., 1990; Braunshofer-Reiter et al., 1998). The nucleos(t)ide analogs are used more frequently than interferon. They inhibit DNA synthesis and suppress viral replication by 4-5 log₁₀ in up to 70-90% patients, often to below the standard clinical detection limit of 300-400 copies/ml (Braunshofer-Reiter et al., 1998; Nowotny et al., 2005; Klumpp et al., 2003. However, treatment eradicates the infection as measured by loss of the viral surface antigen (HBsAg) from the serum in only 3-6% of patients even after years of therapy (Braunshofer-Reiter et al., 1998; Nowotny et al., 2005; Klumpp et al., 2003; Nowotny et al., 2006). Antiviral resistance was a major problem with the earlier nucleos(t)ide analogs, but resistance to the newer drugs entecavir and tenofovir is very low (Parker et al., 2004; Keck et al., 1998; Goedken et al., 2001; Li et al., 1995). This has converted HBV from a steadily worsening disease into a controllable condition for most individuals (McClure, 1993). The cost of this control is indefinite administration of the drugs (probably life-long; (Song et al., 2004), with ongoing expenses of $400-600/month (Poch et al., 1989; Hu et al. 1996; Hu et al., 1997) and unpredictable adverse effects associated with decades-long exposure to the drugs.

As such, there remains a need to develop new therapeutic options for these diseases.

### SUMMARY

Thus, in accordance with the present disclosure, there is provided compounds and methods for inhibiting HBV replication and treating hepatitis B.

In some aspects, the present disclosure provides methods of inhibiting hepatitis B virus replication comprising contacting the hepatitis B virus with an effective amount of a compound of the formula:

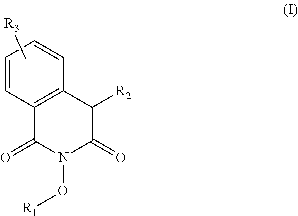

wherein:
R₁ is hydrogen or alkyl_{(C≤8)}, acyl_{(C≤8)}, or a substituted version of either of these groups;
R₂ is alkyl_{(C≤12)}, aryl_{(C≤12)}, aralkyl_{(C≤12)}, or a substituted version of any of these groups; or —C(O)ORₐ or —C(O)NRₐR_{b}, wherein:
   Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
   or a pharmaceutically acceptable salt thereof. In some embodiments, the compound is further defined as:

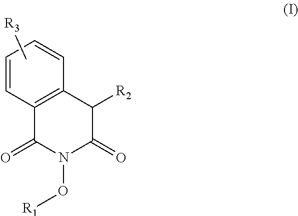

wherein:
R₁ is hydrogen or alkyl_{(C≤8)}, acyl_{(C≤8)}, or a substituted version of either of these groups;
R₂ is —C(O)ORₐ or —C(O)NRₐR_{b}, wherein:
   Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
   or a pharmaceutically acceptable salt thereof. In some embodiments, the compound is further defined as:

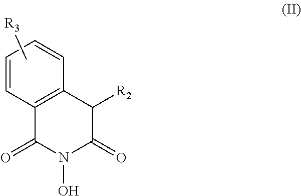

wherein:
R₂ is —C(O)ORₐ or —C(O)NRₐR_{b}, wherein:
   Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
   or a pharmaceutically acceptable salt thereof.

In some embodiments, R₂ is —C(O)ORₐ, wherein: Rₐ is hydrogen or alkyl_{(C≤8)}, aryl_{(C≤8)}, aralkyl_{(C≤8)}, or a substituted version of any of these groups. In some embodiments, Rₐ is alkyl_{(C≤8)} or substituted alkyl_{(C≤8)} such as methyl. In other embodiments, R₂ is —C(O)NRₐR_{b}, wherein: Rₐ and R_{b} is hydrogen or alkyl_{(C≤8)}, aryl_{(C≤8)}, aralkyl_{(C≤8)}, or a substituted version of any of these groups. In some embodiments, Rₐ is hydrogen. In other embodiments, Rₐ is alkyl_{(C≤8)} or substituted alkyl_{(C≤8)} such as ethyl. In other embodiments, Rₐ is aryl_{(C≤8)} or substituted aryl_{(C≤8)} such as phenyl. In other embodiments, Rₐ is aralkyl_{(C≤8)} or substituted aralkyl_{(C≤8)} such as benzyl, 4-fluorobenzyl, or 4-methoxybenzyl. In some embodiments, R_{b} is hydrogen. In other embodiments, R_{b} is alkyl_{(C≤8)} or substituted alkyl_{(C≤8)} such as ethyl.

In some embodiments, R₃ is hydrogen. In other embodiments, R₃ is halo such as chloro. In other embodiments, R₃ is nitro. In other embodiments, R₃ is alkoxy_{(C≤8)} or substituted alkoxy_{(C≤8)} such as methoxy. In other embodiments, R₃ is acyl_{(C≤8)} or substituted acyl_{(C≤8)}. In other embodiments, R₃ is —NHC(O)Ph. In some embodiments, the compound is further defined as:

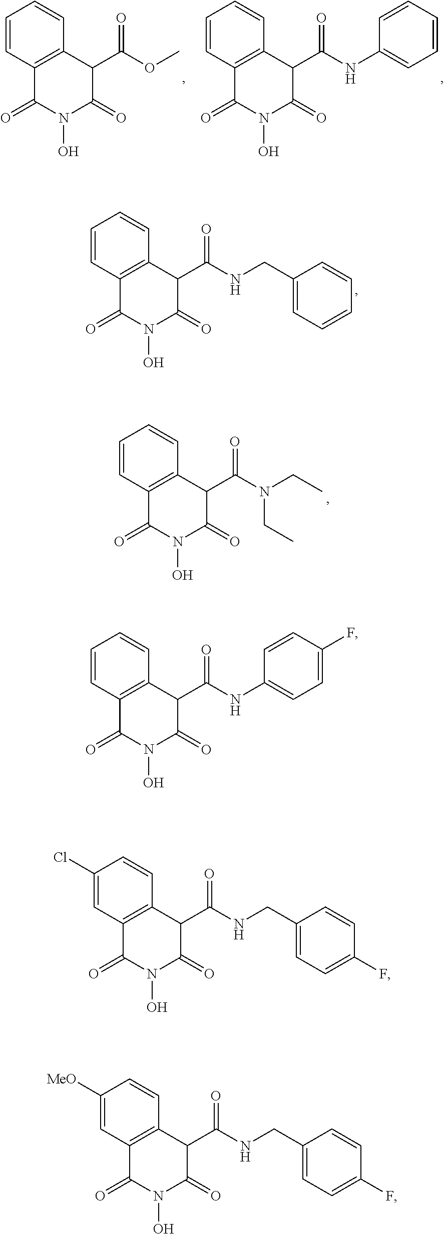

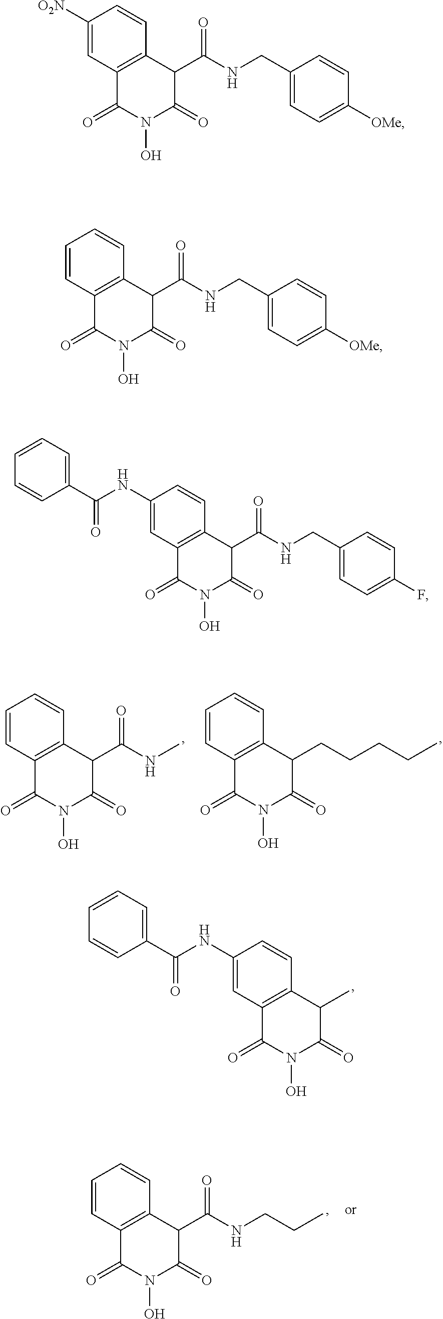

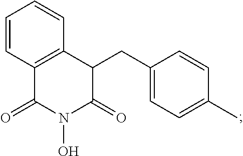 [SMILES: Cc1ccc(CC2C(=O)N(O)C(=O)c3ccccc32)cc1]

or a pharmaceutically acceptable salt thereof.

In some embodiments, the method is preformed in vivo. In other embodiments, the method is preformed ex vivo. In other embodiments, the method is preformed in vitro. In some embodiments, the method is sufficient to treat an infection of a hepatitis B virus.

In yet another aspect, the present disclosure provides methods of treating a hepatitis B virus infection in a patient comprising administering to the patient in need thereof a pharmaceutically effective amount of a compound of the formula:

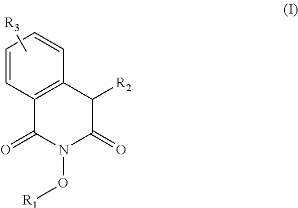

wherein:
R₁ is hydrogen or alkyl_{(C≤8)}, acyl_{(C≤8)}, or a substituted version of either of these groups;
R₂ is alkyl_{(C≤12)}, aryl_{(C≤12)}, aralkyl_{(C≤12)}, or a substituted version of any of these groups; or —C(O)ORₐ or —C(O)NRₐR_{b}, wherein:
   Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
   or a pharmaceutically acceptable salt thereof. In some embodiments, the compound is further defined as:

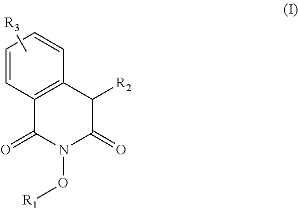

wherein:
R₁ is hydrogen or alkyl_{(C≤8)}, acyl_{(C≤8)}, or a substituted version of either of these groups;
R₂ is —C(O)ORₐ or —C(O)NRₐR_{b}, wherein:
   Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
   or a pharmaceutically acceptable salt thereof. In some embodiments, the compound is further defined as:

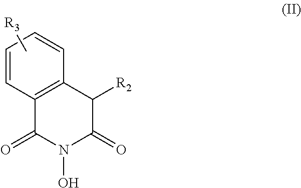

wherein:
R₂ is —C(O)ORₐ or —C(O)NRₐR_{b}, wherein:
   Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};

or a pharmaceutically acceptable salt thereof.

In some embodiments, R₂ is —C(O)ORₐ, wherein: Rₐ is hydrogen or alkyl_{(C≤8)}, aryl_{(C≤8)}, aralkyl_{(C≤8)}, or a substituted version of any of these groups. In some embodiments, Rₐ is alkyl_{(C≤8)} or substituted alkyl_{(C≤8)} such as methyl. In other embodiments, R₂ is —C(O)NRₐR_{b}, wherein: Rₐ and R_{b} is hydrogen or alkyl_{(C≤8)}, aryl_{(C≤8)}, aralkyl_{(C≤8)}, or a substituted version of any of these groups. In some embodiments, Rₐ is hydrogen. In other embodiments, Rₐ is alkyl_{(C≤8)} or substituted alkyl_{(C≤8)} such as ethyl. In other embodiments, Rₐ is aryl_{(C≤8)} or substituted aryl_{(C≤8)} such as phenyl. In other embodiments, Rₐ is aralkyl_{(C≤8)} or substituted aralkyl_{(C≤8)} such as benzyl, 4-fluorobenzyl, or 4-methoxybenzyl. In some embodiments, R_{b} is hydrogen. In other embodiments, R_{b} is alkyl_{(C≤8)} or substituted alkyl_{(C≤8)} such as ethyl.

In some embodiments, R₃ is hydrogen. In other embodiments, R₃ is halo such as chloro. In other embodiments, R₃ is nitro. In other embodiments, R₃ is alkoxy_{(C≤8)} or substituted alkoxy_{(C≤8)} such as methoxy. In other embodiments, R₃ is acyl_{(C≤8)} or substituted acyl_{(C≤8)}. In other embodiments, R₃ is —NHC(O)Ph. In some embodiments, the compound is further defined as:

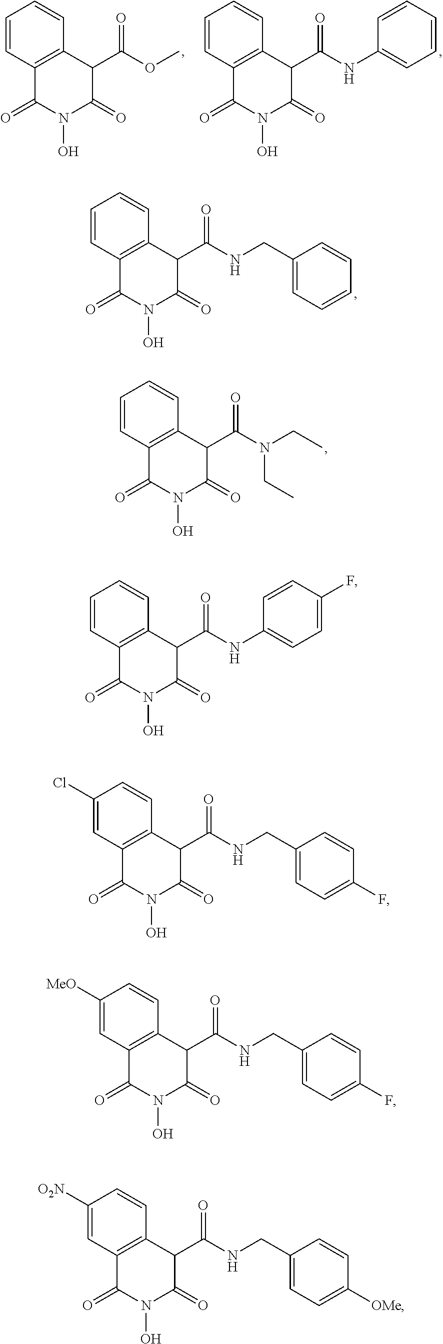

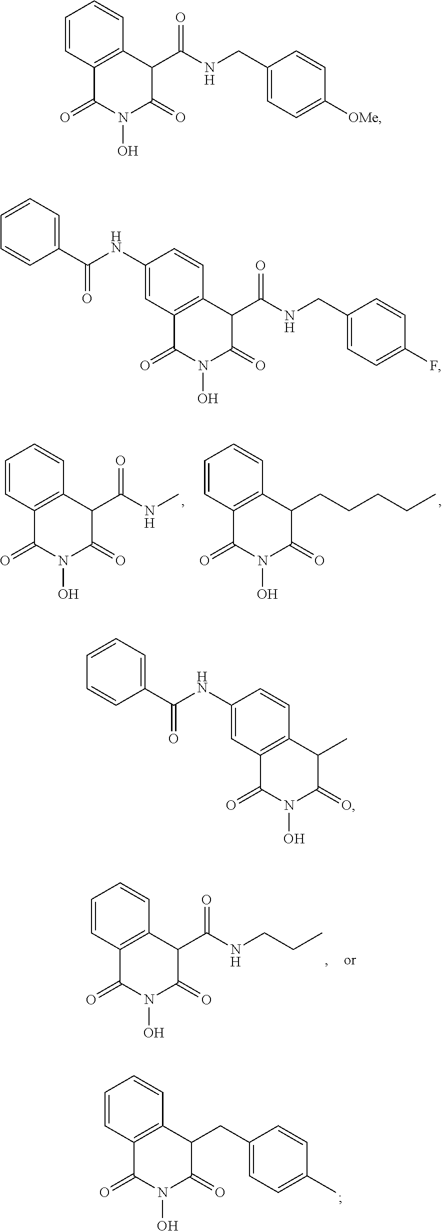

or a pharmaceutically acceptable salt thereof.

In some embodiments, the patient is a mammal such as a human. In some embodiments, the methods further comprise a second antiviral therapy. In some embodiments, the second antiviral therapy is interferon alfa-2b, lamivudine, adefovir, telbivudine, entercavir, or tenofovir.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description.

FIGS. 1A & 1B show the RNAseH inhibitors work synergistically with Lamivudine against HBV replication. Chou-Talaly combination indexes for compounds #1 (FIG. 1A) and #46 (FIG. 1B) together with Lamivudine. Additive interactions are shown with the red line, synergistic interactions below the line, and antagonistic interactions are above the line.

FIGS. 2A & 2B show that HBV's genetic variation is unlikely to present a barrier to RNAseH drug development. Four variant patient-derived RNAseH enzymes were expressed as recombinant enzymes, purified, and tested in an RNAseH assay with compounds #1 (FIG. 2A) and #46 (FIG. 2B) at their respective IC₅₀s.

FIG. 3 shows the structure of the compounds used herein.

FIG. 4 shows the representative replication inhibition and cytotoxicity experiments. Replication inhibition by HIDs and an HPD was measured against an HBV genotype D isolate in HepDES19 cells and plotted against compound concentration (log₁₀ [M]).

Cytotoxicity was assessed by MTS, NR, and LDH assays and plotted against compound concentration (log₁₀ [M]). EC₅₀ experiments were done with a single replicate per condition and CC₅₀ assays were each conducted in triplicate. EC₅₀ values were calculated based on the decline of the plus-polarity DNA strand; positive-polarity DNAs are in black; negative-polarity DNAs are in grey. EC₅₀ and CC₅₀ values are reported as the average of two or three independent experiments±one standard deviation.

FIG. 5 shows the effects of the compounds on HBV DNA elongation. Isolated HBV capsids were incubated with radiolabeled dNTPs and compounds at 4× their EC₅₀s to measure DNA elongation by the encapsidated viral reverse transcriptase in an EPR reactions. Nucleic acids were harvested following the EPR reaction, resolved by electrophoresis, and detected by autoradiography.

FIG. 6 shows the detection of capsid-associated RNA:DNA heteroduplexes. HepDES19 cells replicating HBV were treated with 4×EC₅₀ of the HID compound #89, 30 μM of the inactive compound #138, or with DMSO as a vehicle control. HBV capsids were harvested and encapsidated nucleic acids were purified. The nucleic acids were split into two pools, one that was mock treated and the other which was treated with RNaseA. Nucleic acids were resolved by electrophoresis and HBV sequences were detected by Southern analysis. RNA:DNA heteroduplex accumulation was determined as a collapse in migration patterns in the RNaseA-treated samples. rcDNA, relaxed circular DNA; DS DNA, double-stranded DNAs; ssDNA, single-stranded DNAs.

FIGS. 7A & 7B shows the preliminary structure activity relationship (SAR) from the compounds analyzed herein. FIG. 7A shows the minimal pharmacophore shared by all active compounds. FIG. 7B shows the preliminary SAR for one class of the HID compounds.

FIGS. 8A-8H shows the effects of the compounds on purified HBV RNaseH. (FIG. 8A) HBV RNaseH inhibition was determined using a molecular beacon RNaseH assay in which quenching of fluorescence from the RNaseH substrate is measured following removal of the RNA strand by the RNaseH, causing the folding of the DNA strand into a hairpin. (FIG. 8B) Activity of wild-type and an active site knockout mutant (D702A/E731A) HBV RNaseHs. (FIGS. 8C-8H) Fluorescence intensity from representative RNaseH reactions incubated for 60 min in the presence of 0 to 500 μM of compounds #1, 81, 83, 138, 208, and 211.

### DETAILED DESCRIPTION

The inventors have previously demonstrated that inhibitors of nucleotidyl-transferase superfamily (NTS) enzymes can come from multiple different chemical classes. The compounds described herein may be used for a variety of different viral infections including the hepatitis B virus. Inhibitors of NTS enzymes like this compound may well have a high barrier to development of antiviral resistance, and its unique mode of action suggests that it should be a good candidate for combination therapy with the existing antiviral drugs to improve overall efficacy of antiviral therapy. These and other aspects of the disclosure are discussed in detail below.

### A. HEPATITIS B VIRUS

1. Biology

Hepatitis B virus, abbreviated HBV, is a species of the genus Orthohepadnavirus, which is likewise a part of the Hepadnaviridae family of viruses. This virus causes the disease hepatitis B. In addition to causing hepatitis B, infection with HBV can lead to hepatic fibrosis, cirrhosis and hepatocellular carcinoma. It has also been suggested that it may increase the risk of pancreatic cancer.

The hepatitis B virus is classified as the type species of the Orthohepadnavirus, which contains at least five other species: the pomona roundleaf bat hepatitis virus, long-fingered bat hepatitis virus, the Ground squirrel hepatitis virus, Woodchuck hepatitis virus, and the Woolly monkey hepatitis B virus. The genus is classified as part of the Hepadnaviridae family along with Avihepadnavirus. This family of viruses have not been assigned to a viral order. Viruses similar to hepatitis B have been found in all the Old World apes (orangutan, gibbons, gorillas and chimpanzees) and from a New World woolly monkey suggesting an ancient origin for this virus in primates.

The virus is divided into four major serotypes (adr, adw, ayr, ayw) based on antigenic epitopes present on its envelope proteins, and into eight genotypes (A-H) according to overall nucleotide sequence variation of the genome. The genotypes have a distinct geographical distribution and are used in tracing the evolution and transmission of the virus. Differences between genotypes affect the disease severity, course and likelihood of complications, and response to treatment and possibly vaccination.

Hepatitis B virus is a member of the Hepadnavirus family. The virus particle (virion) consists of an outer lipid envelope and an icosahedral nucleocapsid core composed of protein. The nucleocapsid encloses the viral DNA and a DNA polymerase that has reverse transcriptase activity similar to retroviruses. The outer envelope contains embedded proteins which are involved in viral binding of, and entry into, susceptible cells. The virus is one of the smallest enveloped animal viruses with a virion diameter of 42 nm, but pleomorphic forms exist, including filamentous and spherical bodies that both lack a core. These particles are not infectious and are composed of the lipid and protein that forms part of the surface of the virion, which is called the surface antigen (HBsAg), and are produced in excess during the life cycle of the virus. The HBV virus itself is called a Dane particle and consists of HBsAg, the core protein (HBcAg) and the Hepatitis B virus DNA polymerase. The functions of the small regulatory protein (HBx) are not yet well known but may be related to interfering with transcription, signal transduction, signal transduction, cell cycle progress, protein degradation, apoptosis, or chromosomal stability. The virus also produces a secreted protein called HBeAg that is an amino-terminal extension of HBcAg initiating from an upstream start codon that is involved in suppressing antiviral immune responses.

The genome of HBV is made of circular DNA, but it is unusual because the DNA is not fully double-stranded. One end of the full length strand is linked to the viral DNA polymerase. The genome is 3020-3320 nucleotides long (for the full length strand) and 1700-2800 nucleotides long (for the short length strand). The negative-sense, (non-coding), strand is the complete strand and it is complementary to the viral mRNA. The viral DNA is found in the nucleus soon after infection of the cell. The partially double-stranded DNA is rendered fully double-stranded shortly after infection of a cell by completion of the (+) sense strand and removal of a protein molecule from the (−) sense strand and a short sequence of RNA from the (+) sense strand. A short terminal duplication of are removed from the ends of the (−) sense strand and the ends are rejoined. The mature nuclear form of the genome is called the “cccDNA.” The cccDNA is the template for transcription of all of the viral mRNAs.

There are four known genes encoded by the genome called C, X, P, and S. The core protein (HBcAg) is coded for by gene C, and its start codon is preceded by an upstream in-frame AUG start codon from which the pre-core protein is produced. HBeAg is produced by proteolytic processing of the pre-core protein. The DNA polymerase is encoded by gene P. Gene S is the gene that codes for the surface antigens (HBsAg). The HBsAg gene is one long open reading frame but contains three in frame “start” (ATG) codons that divide the gene into three sections, pre-S1, pre-S2, and S. Because of the multiple start codons, polypeptides of three different sizes called large, middle, and small (pre-S1+pre-S2+S, pre-S2+S, or S) are produced. The function of the protein coded for by gene X is not fully understood, but it is known to have pleiotropic regulatory functions in both the cytoplasm and nucleus.

There are at least eight known genotypes labeled A through H. A possible new “I” genotype has been described, but acceptance of this notation is not universal. Different genotypes may respond to treatment in different ways. The genotypes differ by at least 8% of the sequence and have distinct geographical distributions and this has been associated with anthropological history. Type F which diverges from the other genomes by 14% is the most divergent type known. Type A is prevalent in Europe, Africa and South-east Asia, including the Philippines. Type B and C are predominant in Asia; type D is common in the Mediterranean area, the Middle East and India; type E is localized in sub-Saharan Africa; type F (or H) is restricted to Central and South America. Type G has been found in France and Germany. Genotypes A, D and F are predominant in Brazil and all genotypes occur in the United States with frequencies dependent on ethnicity. The E and F strains appear to have originated in aboriginal populations of Africa and the New World, respectively. Within these genotypes, 24 subtypes have been described which differ by 4-8% of the genome:
Type A has two subtypes: Aa (A1) in Africa/Asia and the Philippines and Ae (A2) in Europe/United States.
Type B has two distinct geographical distributions: Bj/B1 (‘j’—Japan) and Ba/B2 (‘a’—Asia). Type Ba has been further subdivided into four clades (B2-B4).
Type C has two geographically subtypes: Cs (C1) in South-east Asia and Ce (C2) in East Asia. The C subtypes have been divided into five clades (C1-C5). A sixth clade (C6) has been described in the Philippines but only in one isolate to date. Type C1 is associated with Vietnam, Myanmar and Thailand; type C2 with Japan, Korea and China; type C3 with New Caledonia and Polynesia; C4 with Australia; and C5 with the Philippines. A further subtype has been described in Papua, Indonesia.
Type D has been divided into 7 subtypes (D1-D7).
Type F has been subdivided into 4 subtypes (F1-F4). F1 has been further divided in to 1a and 1b. In Venezuela subtypes F1, F2, and F3 are found in East and West Amerindians. Among South Amerindians only F3 was found. Subtypes Ia, III, and IV exhibit a restricted geographic distribution (Central America, the North and the South of South America respectively) while clades Ib and II are found in all the Americas except in the Northern South America and North America respectively.

The life cycle of hepatitis B virus is complex. Hepatitis B is one of a few known non-retroviral viruses which use reverse transcription as a part of its replication process:
Attachment—The virus gains entry into the cell by binding to a receptor on the surface of the cell and enters it by endocytosis.
Penetration—The virus membrane then fuses with the host cell's membrane releasing the DNA and core proteins into the cytoplasm.
Uncoating—Because the virus multiplies via RNA made by a host enzyme, the viral genomic DNA has to be transferred to the cell nucleus by host proteins. The core proteins dissociate from the partially double-stranded viral DNA is then made fully double-stranded and transformed into covalently closed circular DNA (cccDNA) that serves as a template for transcription of four viral mRNAs.
Replication—The cccDNA is the transcriptional template for all of HBV's RNAs. The largest of the mRNAs is called the pre-core mRNA that encodes HBeAg. A slightly shorter mRNA is called the pregenomic RNA that encodes the HBcAg and the viral DNA polymerase. Both the precore and pregenomic RNAs are longer than the viral genome, but only the pregenomic RNA is packaged into nascent core particles along with the viral polymerase. Reverse transcription within the capsids is catalyzed by the coordinate activity of the viral DNA polymerase's reverse transcriptase and ribonuclease H activities and results in the partially double-stranded viral DNA found within HBV virions. Assembly and Release—Progeny virions are formed budding of the viral capsid particles containing the viral DNA into endoplasmic-reticulum-derived membranes, where they pick up their envelope and HBsAgs are released from the cell by non-cytolytic secretion or are returned to the nucleus and re-cycled to produce even more copies of the nuclear cccDNA.

2. Treatment

Currently, there are seven FDA approved drugs in the U.S. to treat chronic HBV: Intron A® (Interferon Alpha), Pegasys® (Pegylated Interferon), Epivir HBV® (Lamivudine), Hepsera® (Adefovir), Baraclude® (Entecavir), Tyzeka® (Telbivudine), and Viread® (Tenofovir).

Adefovir, previously called bis-POM PMEA, with trade names Preveon® and Hepsera®, is an orally-administered nucleotide analog reverse transcriptase inhibitor (ntRTI). It can be formulated as the pivoxil prodrug adefovir dipivoxil. Adefovir works by blocking reverse transcriptase, the enzyme that is crucial for the hepatitis B virus (HBV) to reproduce in the body because it synthesizes the viral DNA. It is approved for the treatment of chronic hepatitis B in adults with evidence of active viral replication and either evidence of persistent elevations in serum aminotransferases (primarily ALT) or histologically active disease. The main benefit of adefovir over drugs like lamivudine (below) is that it takes a much longer period of time before the virus develops resistance to it. Adefovir dipivoxil contains two pivaloyloxymethyl units, making it a prodrug form of adefovir.

Lamivudine (2′,3′-dideoxy-3′-thiacytidine, commonly called 3TC) is a potent nucleoside analog reverse transcriptase inhibitor (nRTI). It is marketed by GlaxoSmithKline with the brand names Zeffix®, Heptovir®, Epivir®, and Epivir-HBV®. Lamivudine has been used for treatment of chronic hepatitis B at a lower dose than for treatment of HIV. It improves the seroconversion of HBeAg positive hepatitis B and also improves histology staging of the liver. Long term use of lamivudine unfortunately leads to emergence of a resistant hepatitis B virus (YMDD) mutant. Despite this, lamivudine is still used widely as it is well tolerated and as it is less expensive than the newer drugs and is the only anti-HBV drug many people in emerging economies can afford.

Lamivudine is an analogue of cytidine. It can inhibit both types (1 and 2) of HIV reverse transcriptase and also the reverse transcriptase of hepatitis B. It is phosphorylated to active metabolites that compete for incorporation into viral DNA. It inhibits the HIV reverse transcriptase enzyme competitively and acts as a chain terminator of DNA synthesis. The lack of a 3′-OH group in the incorporated nucleoside analogue prevents the formation of the 5′ to 3′ phosphodiester linkage essential for DNA chain elongation, and therefore, the viral DNA growth is terminated. Lamivudine is administered orally, and it is rapidly absorbed with a bio-availability of over 80%. Some research suggests that lamivudine can cross the blood-brain barrier.

Entecavir, abbreviated ETV, is an oral antiviral drug used in the treatment of hepatitis B infection. It is marketed under the trade names Baraclude® (BMS) and Entaliv® (DRL).

Entecavir is a nucleoside analog (more specifically, a guanosine analogue) that inhibits reverse transcription and DNA replication thus preventing transcription in the viral replication process. The drug's manufacturer claims that entecavir is more efficacious than previous agents used to treat hepatitis B (lamivudine and adefovir). Entecavir was approved by the U.S.FDA in March 2005 and is used to treat chronic hepatitis B. It also helps prevent the hepatitis B virus from multiplying and infecting new liver cells. Entecavir is also indicated for the treatment of chronic hepatitis B in adults with HIV/AIDS infection. However, entecavir is not active against HIV.

Telbivudine is an antiviral drug used in the treatment of hepatitis B infection. It is marketed by Swiss pharmaceutical company Novartis under the trade names Sebivo® (Europe) and Tyzeka® (United States). Clinical trials have shown it to be significantly more effective than lamivudine or adefovir, and less likely to cause resistance. Telbivudine is a synthetic thymidine nucleoside analogue; it is the L-isomer of thymidine. It is taken once daily.

Tenofovir disoproxil fumarate (TDF or PMPA), marketed by Gilead Sciences under the trade name Viread®, it is also a nucleotide analogue reverse transcriptase inhibitor (nRTIs) which blocks the HBV reverse transcriptase, an enzyme crucial to viral production. Tenofovir disoproxil fumarate is a prodrug form of tenofovir. Tenofovir is indicated in combination with other antiretroviral agents for the treatment of HIV-1 infection in adults. This indication is based on analyses of plasma HIV-1 RNA levels and CD4 cell counts in controlled studies of tenofovir in treatment-naive and treatment-experienced adults. There are no study results demonstrating the effect of tenofovir on the clinical progression of HIV. It also has activity against wild-type and lamivudine-resistant HBV.

### B. NUCLEOTIDYL TRANSFERASE SUPERFAMILY ENZYMES

The inhibitors screened in this project were selected for their ability to inhibit the HIV RNAse H and/or integrase enzymes (or to be close chemical analogs of known inhibitors). The RNAse H and integrase are members of the nucleotidyl transferase superfamily (NTS) whose members share a similar protein fold and enzymatic mechanisms (Yang 1995). RNAse H enzymes (Hostomsky et al., 1993a; 1993b; 1993c) digest RNA when it is hybridized to DNA. Their physiological roles include removal of RNA primers during DNA synthesis, removal of abortive transcription products, and removal of RNA strands following reverse transcription by viruses or retrotransposons. Integrase enzymes cleave DNA strands and catalyze the covalent insertion of another DNA strand at the cleavage site. Without wishing to be bound by any theory, it is believe that the mechanism of action for the inhibitors claimed here is by inhibiting the HBV RNase H.

The NTS family of enzymes includes E. coli RNase H I and II (Katayanagi et al., 1990, Yang et al., 1990 and Lai et al., 2000); human RNase H 1 and 2 (Lima et al., 2001, Frank et al., 1998 and Frank et al., 1998); the RuvC Holiday junction resolvase (Ariyoshi et al., 1994); and the Argonaute RNAse (Parker et al., 2004 and Song et al., 2004); retroviral RNase H enzymes including the HIV enzyme (Nowotny 2009); retroviral integrases including the HIV integrase (Dyda et al., 1994); and the hepatitis B virus (HBV) RNase H (Tavis et al., 2013). These enzymes function in a wide range of nucleic acid metabolic events, including RNA and DNA digestion, DNA recombination, DNA integration, DNA excision, replication fork repair, DNA repair, miRNA maturation, and miRNA-directed RNA cleavage. The canonical RNase H structure contains about 100 amino acids that fold into a 5-stranded β-sheet overlaid with 3 α-helices arranged like an “H”. Within the active site are four conserved carboxylates (the “DEDD” motif) that coordinate two divalent cations (Nowotny et al., 2005).

The RNase H enzymatic mechanism is believed to involve both divalent cations (Klumpp et al., 2003; Yang and Steitz, 1995), although a 1-ion mechanism has been proposed (Goedken and Marqusee, 2001; Keck et al., 1998). There are 3 classes of RNAse Hs distinguished by how they bind to their substrates. RNA binding by the “stand-alone” class typified by E. coli RNAse H I is promoted by a basic “handle” region (Hostomsky et al., 1993; Kwun et al., 2001). Eukaryotic RNase Hs typically contain a “RHBD” domain that influences nucleic acid binding. Finally, substrate binding by the retroviral enzymes can either be a property of the RNase H domain itself (e.g., Moloney murine leukemia virus) or may require the reverse transcriptase domain to provide sufficient affinity for the nucleic acid substrate (e.g., HIV) (Hostomsky et al., 1993; Smith et al., 1994).

The HBV RNase H is a NTS enzyme. Mutational analysis of the HBV RNase H revealed the DEDD active site residues to be D702, E731, D750, and D790 (numbering for HBV strain adw2) (Gerelsaikhan et al., 1996; Tavis et al., 2013).

HIV reverse transcription requires a virally encoded RNase H activity to remove the viral RNA after it has been copied into DNA. Consequently, the HIV RNase H activity has attracted much attention as a drug target (Billamboz et al., 2011; Bokesch et al., 2008; Budihas et al., 2005; Chung et al., 2011; Chung et al., 2010; Di et al., 2010; Didierjean et al., 2005; Fuji et al., 2009; Himmel et al., 2009; Himmel et al., 2006; Kirschberg et al., 2009; Klarmann et al., 2002; Klumpp et al., 2003; Klumpp and Mirzadegan, 2006; Shaw-Reid et al., 2003; Su et al., 2010; Takada et al., 2007; Wendeler et al., 2008; Williams et al., 2010). Over 100 anti-HIV RNase H compounds, based on a wide variety of chemical scaffolds, have been reported (Chung et al., 2011; Klumpp and Mirzadegan, 2006). They typically have inhibitory concentration-50% (IC₅₀) values in the low M range. The large majority of these compounds inhibit the RNase H by chelating divalent cations in the active site (Billamboz et al., 2011; Chung et al., 2011; Fuji et al., 2009; Himmel et al., 2009; Kirschberg et al., 2009; Su et al., 2010), but compounds that alter the enzyme's conformation or its interaction with nucleic acids have also been reported (Himmel et al., 2006; Wendeler et al., 2008). The inhibitors typically have EC₅₀ values ˜10× higher than the IC₅₀ values, and they often cause modest cytotoxicity, leading to therapeutic indexes (TI) that are usually <10. Second-generation inhibitors with substantially improved efficacy have been reported, (Billamboz et al., 2011; Chung et al., 2011; Williams et al., 2010), and compounds with efficacy and TI values appropriate for a human drug exist (Himmel et al., 2006; Williams et al., 2010).

None of the anti-HIV RNase H compounds have entered clinical trials yet. This is due in part to their relatively low TI values but also to the large number of approved and developmental anti-HIV drugs, raising doubts about the marketability of anti-HIV RNase H compounds. Despite these challenges, the HIV RNase H remains a target of intensive ongoing drug development, as is evidenced by the large number of groups working in the field (Billamboz et al., 2011; Bokesch et al., 2008; Budihas et al., 2005; Chung et al., 2011; Chung et al., 2010; Di et al., 2010; Didierjean et al., 2005; Fuji et al., 2009; Himmel et al., 2009; Himmel et al., 2006; Kirschberg et al., 2009; Klarmann et al., 2002; Klumpp et al., 2003; Klumpp and Mirzadegan, 2006; Shaw-Reid et al., 2003; Su et al., 2010; Takada et al., 2007; Wendeler et al., 2008; Williams et al., 2010).

Because both the RNase H and integrase are NTS enzymes, some anti-RNase H compounds can inhibit the HIV integrase, and some anti-integrase compounds can inhibit the RNase H (Klarmann et al., 2002, Williams et al., 2010 and Billamboz et al., 2011). Despite this cross-inhibitory potential, resistance mutations to HIV DNA polymerase or integrase drugs have not led to cross-resistance to RNase H inhibitors (Billamboz et al., 2011 and Himmel et al., 2006).

HBV reverse transcription requires two viral enzymatic activities that are both located on the viral reverse transcriptase protein. The DNA polymerase activity synthesizes new DNA and is targeted by the nucleos(t)ide analogs. The RNase H destroys the viral RNA after it has been copied into DNA. Inhibiting the RNAse H would block DNA synthesis and consequently halt viral replication, but anti-HBV RNase H drugs have not been developed because enzyme suitable for drug screening could not be readily made. One of the inventors recently produced active recombinant HBV RNase H and identified 35 inhibitors of the RNase H (Table 1; Tavis et al., 2013; Hu et al., 2013; Tavis and Lomonosova, 2015; Lu, et al., 2015; and Cai, et al., 2014).

### C. CHEMICAL ENTITY

The compound of the disclosure contains one or more asymmetrically-substituted carbon or nitrogen atoms, and may be isolated in optically active or racemic form. Thus, all chiral, diastereomeric, racemic form, epimeric form, and all geometric isomeric forms of the chemical formula are intended, unless the specific stereochemistry or isomeric form is specifically indicated. The compound may occur as a racemate and racemic mixtures, single enantiomers, diastereomeric mixtures and individual diastereomers. In some embodiments, a single enantiomer or diastereomer is obtained. The chiral centers of the compound of the present disclosure can have the S or the R configuration.

Chemical formulas used to represent the compound of the disclosure will typically only show one of possibly several different tautomers. For example, many types of ketone groups are known to exist in equilibrium with corresponding enol groups. Regardless of which tautomer is depicted for a given compound, and regardless of which one is most prevalent, all tautomers of a given chemical formula are intended.

The compound of the disclosure may also have the advantage of being more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical properties over, compounds known in the prior art, whether for use in the indications stated herein or otherwise.

In addition, atoms making up the compound of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of a compound of the present disclosure may be replaced by a sulfur or selenium atom(s).

The compound of the present disclosure may also exist in prodrug form. Since prodrugs are known to enhance numerous desirable qualities of pharmaceuticals (e.g., solubility, bioavailability, manufacturing, etc.), the compound employed in some methods of the disclosure may, if desired, be delivered in prodrug form. Thus, the disclosure contemplates prodrugs of the compound of the present disclosure as well as methods of delivering prodrugs. Prodrugs of the compound employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy group is bonded to any group that, when the prodrug is administered to a subject, cleaves to form a hydroxy group.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

2. Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means ═NH; “cyano” means —CN; “isocyanate” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

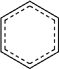

covers, for example,

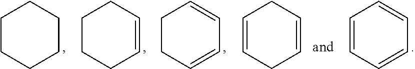

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

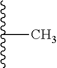

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a group “R” is depicted as a “floating group” on a ring system, for example, in the formula:

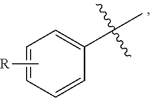

then R may replace any hydrogen atom attached to any of the ring atoms, including a depicted, implied, or expressly defined hydrogen, so long as a stable structure is formed. When a group “R” is depicted as a “floating group” on a fused ring system, as for example in the formula:

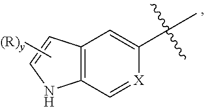

then R may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the group “R” enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” defines the exact number (n) of carbon atoms in the group/class. “C≤n” defines the maximum number (n) of carbon atoms that can be in the group/class, with the minimum number as small as possible for the group/class in question, e.g., it is understood that the minimum number of carbon atoms in the group “alkenyl_{(C≤8)}” or the class “alkene_{(C≤8)}” is two. Compare with “alkoxy_{(C≤10)}”, which designates alkoxy groups having from 1 to 10 carbon atoms. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C5 olefin”, “C5-olefin”, “olefin_{(C5)}”, and “olefin_{C5}” are all synonymous. When any of the chemical groups or compound classes defined herein is modified by the term “substituted”, any carbon atom(s) in a moiety replacing a hydrogen atom is not counted. Thus methoxyhexyl, which has a total of seven carbon atoms, is an example of a substituted alkyl_{(C1-6)}.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” when used without the “substituted” modifier signifies that the compound or chemical group so modified is an acyclic or cyclic, but non-aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” when used to modify a compound or a chemical group refers to a planar unsaturated ring of atoms with 4n+2 electrons in a fully conjugated cyclic 2n system.

The term “alkyl” when used without the “substituted” modifier refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂-(methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

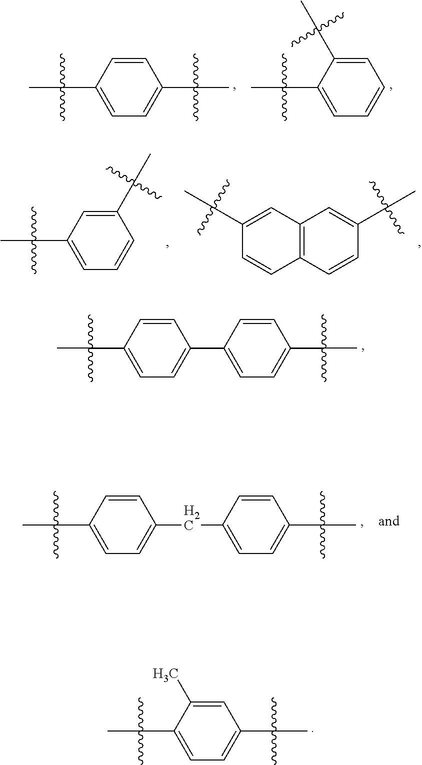

An “arene” refers to the class of compounds having the formula H—R, wherein R is aryl as that term is defined above. Benzene and toluene are non-limiting examples of arenes. When any of these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “aralkyl” when used without the “substituted” modifier refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl. When the term aralkyl is used with the “substituted” modifier one or more hydrogen atom from the alkanediyl and/or the aryl group has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “acyl” when used without the “substituted” modifier refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups.

The term “alkoxy” when used without the “substituted” modifier refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), —OC(CH₃)₃ (tert-butoxy), —OCH(CH₂)₂, —O-cyclopentyl, and —O-cyclohexyl. The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” when used without the “substituted” modifier refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group. When any of these terms is used with the “substituted” modifier one or more hydrogen atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂.

The term “alkylamino” when used without the “substituted” modifier refers to the group —NHR, in which R is an alkyl, as that term is defined above. Non-limiting examples include: —NHCH₃ and —NHCH₂CH₃. The term “dialkylamino” when used without the “substituted” modifier refers to the group —NRR′, in which R and R′ can be the same or different alkyl groups, or R and R′ can be taken together to represent an alkanediyl. Non-limiting examples of dialkylamino groups include: —N(CH₃)₂ and —N(CH₃)(CH₂CH₃). The terms “cycloalkylamino”, “alkenylamino”, “alkynylamino”, “arylamino”, “aralkylamino”, “heteroarylamino”, “heterocycloalkylamino”, “alkoxyamino”, and “alkylsulfonylamino” when used without the “substituted” modifier, refers to groups, defined as —NHR, in which R is cycloalkyl, alkenyl, alkynyl, aryl, aralkyl, heteroaryl, heterocycloalkyl, alkoxy, and alkylsulfonyl, respectively. A non-limiting example of an arylamino group is —NHC₆H₅. The term “amido” (acylamino), when used without the “substituted” modifier, refers to the group —NHR, in which R is acyl, as that term is defined above. A non-limiting example of an amido group is —NHC(O)CH₃. The term “alkylimino” when used without the “substituted” modifier refers to the divalent group ═NR, in which R is an alkyl, as that term is defined above. When any of these terms is used with the “substituted” modifier one or more hydrogen atom attached to a carbon atom has been independently replaced by —OH, —F, —Cl, —Br, —I, —NH₂, —NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects, or +/−5% of the stated value.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to a subject or patient for treating a disease, is sufficient to effect such treatment for the disease.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living vertebrate organism, such as a human, monkey, cow, sheep, goat, dog, cat, mouse, rat, guinea pig, bird, fish or transgenic species thereof. In certain embodiments, the patient or subject is a primate. Non-limiting examples of human subjects are adults, juveniles, infants and fetuses.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of the compound of the present disclosure which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease, including reactivation.

“Prodrug” means a compound that is convertible in vivo metabolically into an inhibitor according to the present disclosure. The prodrug itself may or may not also have activity with respect to a given target protein. For example, a compound comprising a hydroxy group may be administered as an ester that is converted by hydrolysis in vivo to the hydroxy compound. Suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diasteromers can be resolved or separated using techniques known in the art. It is contemplated that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Effective amount,” “therapeutically effective amount” or “pharmaceutically effective amount” means that amount which, when administered to a subject or patient for treating a disease, is sufficient to effect such treatment for the disease.

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease in a subject or patient that is experiencing or displaying the pathology or symptomatology of the disease. In some embodiments, treatment of a patient afflicted with one of the pathological conditions described herein comprises administering to such a patient an amount of compound described herein which is therapeutically effective in controlling the condition or in prolonging the survivability of the patient beyond that expected in the absence of such treatment. As used herein, the term “inhibition” of the condition also refers to slowing, interrupting, arresting or stopping the condition and does not necessarily indicate a total elimination of the condition. It is believed that prolonging the survivability of a patient, beyond being a significant advantageous effect in and of itself, also indicates that the condition is beneficially controlled to some extent.

The above definitions supersede any conflicting definition in any reference that is incorporated by reference herein. The fact that certain terms are defined, however, should not be considered as indicative that any term that is undefined is indefinite. Rather, all terms used are believed to describe the disclosure in terms such that one of ordinary skill can appreciate the scope and practice the present disclosure.

### D. THERAPEUTIC METHODS

1. Pharmaceutical Formulations

In particular embodiments, where clinical application of an active ingredient is undertaken, it will be necessary to prepare a pharmaceutical composition appropriate for the intended application. Generally, this will entail preparing a pharmaceutical composition that is essentially free of pyrogens, as well as any other impurities or contaminants that could be harmful to humans or animals. One also will generally desire to employ appropriate buffers to render the complex stable and allow for uptake by target cells.

Aqueous compositions of the present disclosure comprise an effective amount of the active compound, as discussed above, further dispersed in pharmaceutically acceptable carrier or aqueous medium. Such compositions also are referred to as inocula. The phrase “pharmaceutically or pharmacologically acceptable” refers to compositions that do not produce an adverse, allergic or other untoward reaction when administered to an animal, or a human, as appropriate, as well as the requisite sterility for in vivo uses.

As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients also can be incorporated into the compositions.

Solutions of therapeutic compositions can be prepared in water suitably mixed with a surfactant, such as hydroxypropylcellulose. Dispersions also can be prepared in glycerol, liquid polyethylene glycols, mixtures thereof and in oils. Under ordinary conditions of storage and use, these preparations contain a preservative to prevent the growth of microorganisms.

The therapeutic compositions of the present disclosure are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, a controlled release patch, salve or spray. In some embodiments, the topical formulation by used for administration to the skin, to mucosa membranes such as the eye, eye lids, the genitals, the anus, or the inside of the mouth or nose, and in particular to the cornea.

An effective amount of the therapeutic composition is determined based on the intended goal. The term “unit dose” or “dosage” refers to physically discrete units suitable for use in a subject, each unit containing a predetermined quantity of the therapeutic composition calculated to produce the desired responses, discussed above, in association with its administration, i.e., the appropriate route and treatment regimen. The quantity to be administered, both according to number of treatments and unit dose, depends on the protection desired.

Precise amounts of the therapeutic composition also depend on the judgment of the practitioner and are peculiar to each individual. Factors affecting dose include physical and clinical state of the patient, the route of administration, the intended goal of treatment and the potency, stability and toxicity of the particular therapeutic substance.

2. Routes of Administration

Formulations of the present disclosure are suitable for oral administration. However, the therapeutic compositions of the present disclosure may be administered via any common route so long as the target tissue is available via that route. This includes nasal, buccal, corneal, ocularly, rectal, vaginal, or topical administration, and intradermal, subcutaneous, intramuscular, intraperitoneal or intravenous injection. As such, compositions would be formulated pharmaceutically in route-acceptable compositions that include physiologically acceptable carriers, buffers or other excipients.

As with dosing amounts, the timing of delivery (including intervals and total number of doses) depends on the judgment of the practitioner and are peculiar to each individual. Factors affecting dose include physical and clinical state of the patient, the route of administration, the intended goal of treatment and the potency, stability and toxicity of the particular therapeutic substance.

3. Combination Therapy

In many clinical situations, it is advisable to use a combination of distinct therapies. Thus, it is envisioned that, in addition to the therapies described above, one would also wish to provide to the patient more “traditional” pharmaceutical hepatitis B therapies. Examples of standard therapies are described above. Combinations may be achieved by administering a single composition or pharmacological formulation that includes both agents, or with two distinct compositions or formulations, at the same time, wherein one composition includes the agents of the present disclosure and the other includes the standard therapy. Alternatively, standard therapy may precede or follow the present agent treatment by intervals ranging from minutes to weeks to months. In embodiments where the treatments are applied separately, one would generally ensure that a significant period of time did not expire between the time of each delivery, such that the agents would still be able to exert an advantageously combined effect on the subject. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the agent of the present disclosure, or the standard therapy will be desired. Various combinations may be employed, where the present disclosure compound is “A” and the standard therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are contemplated as well. Additionally, it is contemplated that other antiviral agents such as a pegylated interferon, interferon alfa-2b, lamivudine, adefovir, telbivudine, entercavir, or tenofovir may be used in combination with the compounds described herein.

### E. EXAMPLES

The following examples are included to further illustrate various aspects of the disclosure. It should be appreciated by those of skill in the art that the techniques disclosed in the examples which follow represent techniques and/or compositions discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

1. Materials and Methods

RNaseH expression and purification. Recombinant HBV RNaseH and human RNaseH1 were expressed in E. coli and partially purified by nickel-affinity chromatography as previously described in Tavis, et al. (2013). The enriched extracts were dialyzed into 50 mM HEPES pH 7.3, 300 mM NaCl, 20% glycerol, and 5 mM DTT and stored in liquid nitrogen.

Oligonucleotide-directed RNA cleavage assay. RNaseH activity was measured using an oligonucleotide-directed RNA cleavage assay as previously described in Hu, et al., Tavis, et al., and Cai, et al. (2013; 2013; and 2014, respectfully). Briefly, 6 μL protein extract was mixed on ice with an internally ³²P-labeled 264 nt RNA derived from the Duck Hepatitis B Virus genome (DRF+ RNA) plus 3 μg oligonucleotide D2507- or its inverse-complement oligonucleotide D2526+ as a negative control. This mixture was incubated with test compounds in 50 mM Tris pH 8.0, 190 mM NaCl, 5 mM MgCl₂, 3.5 mM DTT, 0.05% NP40, 6% glycerol, and 1% DMSO at 42° C. for 90 minutes. Cleavage products were resolved by denaturing polyacrylamide gel electrophoresis, detected by autoradiography, and quantified using ImageJ. Non-specific background values were determined from the incorrect oligonucleotide negative control lane and subtracted from all experimental values. IC₅₀ values were then calculated with GraphPad Prism using three-parameter log(inhibitor) vs. response non-linear curve fitting with the curve minimum set to zero to reflect background subtraction.

HBV replication assay. Inhibition of HBV replication was measured in HepDES19 cells as previously described in Cai, et al. (2014). Cells were seeded into 6-well plates and incubated in DMEM/F12, 10% fetal bovine serum (FBS), 1% penicillin/streptomycin (P/S) with 1 μg/mL tetracycline. Tetracycline was withdrawn after 24 hours. The test compound was applied to duplicate wells 48 hours later in medium containing a final DMSO concentration of 1%, and medium containing the compound was refreshed daily for the following two days. Cells were harvested and non-encapsidated nucleic acids were digested with micrococcal nuclease (New England Biolabs). HBV DNA was purified from capsids using QIAamp Cador Pathogen Mini Kit (Qiagen) with proteinase K incubation overnight at 37° C. TaqMan PCR was performed for 40 cycles at an annealing temperature of 60° C. Primers and probe (IDT Inc.) for the plus-polarity strand were: 5′CATGAACAAGAGATGATTAGGCAGAG3′; 5′GGAGGCTGTAGGCATAAATTGG3′; 5′/56-FAM/CTGCGCACC/ZEN/AGCACCATGCA/3IABkFQ. Primers and probe for the minus-polarity strand were: 5′ GCAGATGAGAAGGCACAGA3′; 5′CTTCTCCGTCTGCCGTT3′; 5′/56-FAM/AGTCCGCGT/ZEN/AAAGAGAGGTGCG/3IABkFQ.

MTT cytotoxicity assays. 1.0×10⁴ HepDES19 cells per well were seeded in 96-well plates and incubated in DMEM with 10% FBS plus 1% P/S, 1% non-essential amino acids, and 1% glutamine. Compounds were diluted in medium to the indicated concentrations plus 1% DMSO and added to cells 24 hours after plating, with each concentration tested in triplicate. Medium containing the compound was refreshed daily for the next two days. Thiazolyl blue tetrazolium bromide (MTT, Sigma-Aldrich) was added to 0.25 mg/mL, the cultures were incubated for 60 minutes, metabolites were solubilized in acidic isopropanol, and absorbance was read at 570 nM.

2. HBV Activity

HBV activity of some of the compounds described herein is shown in Table 1 below.

TABLE 1
Compound Activity in Biochemical Assays

| Compound | | HBV | Human |
|---|---|---|---|
| ID | Structure | RNaseH | RNaseH1 |
| 78 | 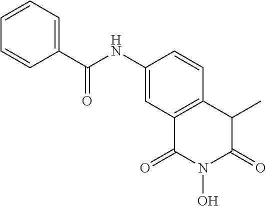 [SMILES: CC1C(=O)N(O)C(=O)c2cc(NC(=O)c3ccccc3)ccc21] | — | >100 |
| 79 | 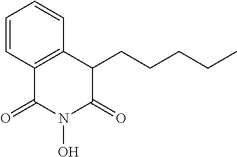 [SMILES: CCCCCC1C(=O)N(O)C(=O)c2ccccc21] | — | >100 |
| 80 | 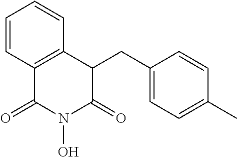 [SMILES: Cc1ccc(CC2C(=O)N(O)C(=O)c3ccccc32)cc1] | — | >100 |
| 81 | 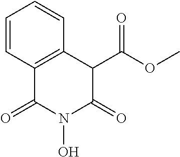 [SMILES: COC(=O)C1C(=O)N(O)C(=O)c2ccccc21] | +/− | 4.9 |
| 82 | 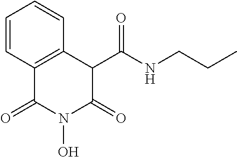 [SMILES: CCCNC(=O)C1C(=O)N(O)C(=O)c2ccccc21] | +/− | +++ |
| 83 | 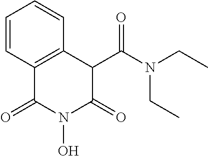 [SMILES: CCN(CC)C(=O)C1C(=O)N(O)C(=O)c2ccccc21] | +/− | 3.4 |
| 84 | 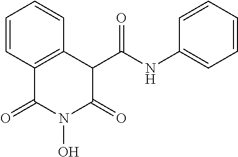 [SMILES: O=C(Nc1ccccc1)C1C(=O)N(O)C(=O)c2ccccc21] | +/− | 3.7 |
| 85 | 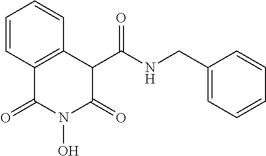 [SMILES: O=C(NCc1ccccc1)C1C(=O)N(O)C(=O)c2ccccc21] | +/− | 3.9 |
| 86 | 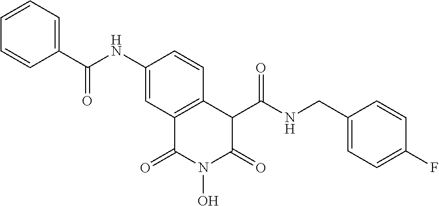 [SMILES: O=C(Nc1ccc2c(c1)C(=O)N(O)C(=O)C2C(=O)NCc1ccc(F)cc1)c1ccccc1] | +/− | 4.0 |
| 87 | 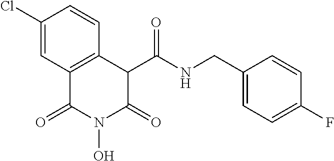 [SMILES: O=C(NCc1ccc(F)cc1)C1C(=O)N(O)C(=O)c2cc(Cl)ccc21] | — | +++ |
| 88 | 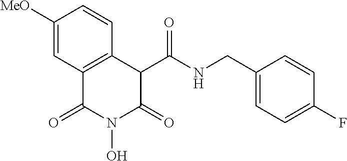 [SMILES: COc1ccc2c(c1)C(=O)N(O)C(=O)C2C(=O)NCc1ccc(F)cc1] | — | +++ |
| 89 | 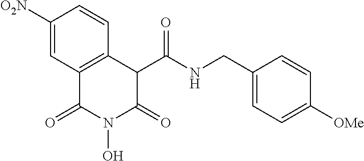 [SMILES: COc1ccc(CNC(=O)C2C(=O)N(O)C(=O)c3cc([N+](=O)[O-])ccc32)cc1] | — | ++ |
| 90 | 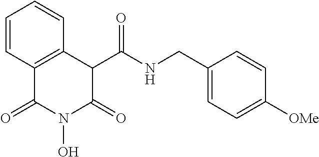 [SMILES: COc1ccc(CNC(=O)C2C(=O)N(O)C(=O)c3ccccc32)cc1] | — | ++ |
| 91 | 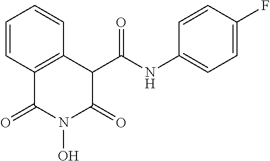 [SMILES: O=C(Nc1ccc(F)cc1)C1C(=O)N(O)C(=O)c2ccccc21] | — | 8.7 |

+/− RNAse H inhibition activity slightly above detection threshold.
+++, detectable inhibition at 10 μM
++, detectable inhibition at 20 μM
+, detectable inhibition at 60 μM
Quantitative values are in μM
ND, not determined

TABLE 2
EC₅₀ and CC₅₀ of Compounds

| Compound | | EC₅₀ | CC₅₀ |
|---|---|---|---|
| ID | Structure | (μM) | (μM) |
| 78 | 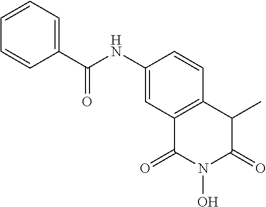 [SMILES: CC1C(=O)N(O)C(=O)c2cc(NC(=O)c3ccccc3)ccc21] | — | ND |
| 79 | 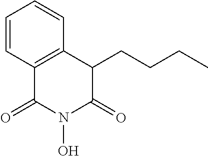 [SMILES: CCCCC1C(=O)N(O)C(=O)c2ccccc21] | — | ND |
| 80 | 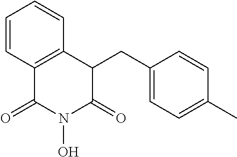 [SMILES: Cc1ccc(CC2C(=O)N(O)C(=O)c3ccccc32)cc1] | — | ND |
| 81 | 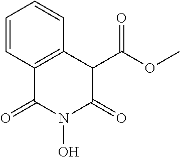 [SMILES: COC(=O)C1C(=O)N(O)C(=O)c2ccccc21] | 4.0 | 57 |
| 82 | 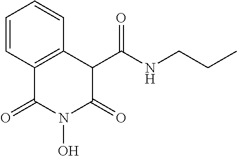 [SMILES: CCCNC(=O)C1C(=O)N(O)C(=O)c2ccccc21] | — | ND |
| 83 | 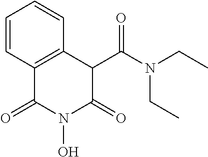 [SMILES: CCN(CC)C(=O)C1C(=O)N(O)C(=O)c2ccccc21] | * | >100 |
| 84 | 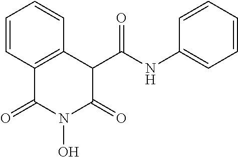 [SMILES: O=C(Nc1ccccc1)C1C(=O)N(O)C(=O)c2ccccc21] | * | 23 |
| 85 | 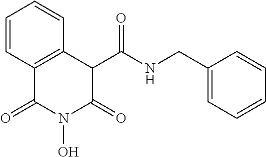 [SMILES: O=C(NCc1ccccc1)C1C(=O)N(O)C(=O)c2ccccc21] | 6.5 | 29 |
| 86 | 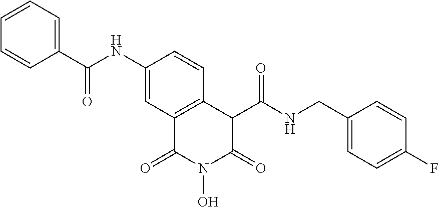 [SMILES: O=C(Nc1ccc2c(c1)C(=O)N(O)C(=O)C2C(=O)NCc1ccc(F)cc1)c1ccccc1] | 1.4 | >100 |
| 87 | 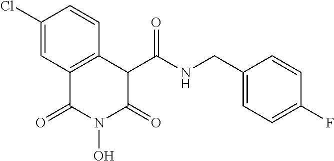 [SMILES: O=C(NCc1ccc(F)cc1)C1C(=O)N(O)C(=O)c2cc(Cl)ccc21] | 1.6 | 37 |
| 88 | 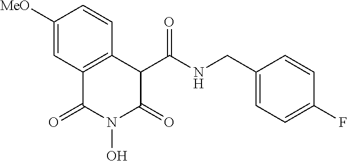 [SMILES: COc1ccc2c(c1)C(=O)N(O)C(=O)C2C(=O)NCc1ccc(F)cc1] | * | 40 |
| 89 | 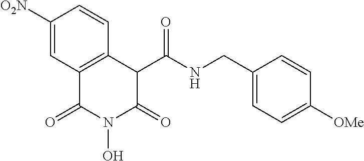 [SMILES: COc1ccc(CNC(=O)C2C(=O)N(O)C(=O)c3cc([N+](=O)[O-])ccc32)cc1] | ** | 28 |
| 90 | 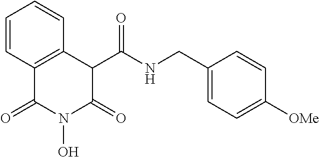 [SMILES: COc1ccc(CNC(=O)C2C(=O)N(O)C(=O)c3ccccc32)cc1] | 18 | 88 |
| 91 | 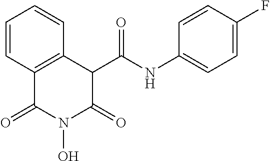 [SMILES: O=C(Nc1ccc(F)cc1)C1C(=O)N(O)C(=O)c2ccccc21] | ** | 11 |

** + DNA suppression <25% and − DNA suppression >60%
* + DNA suppression <50% of − DNA suppression
—, does not suppress HBV DNA replication at 20 μM

3. Synergistic Activity of HBV RNAseH with Nucleoside Analog Drug

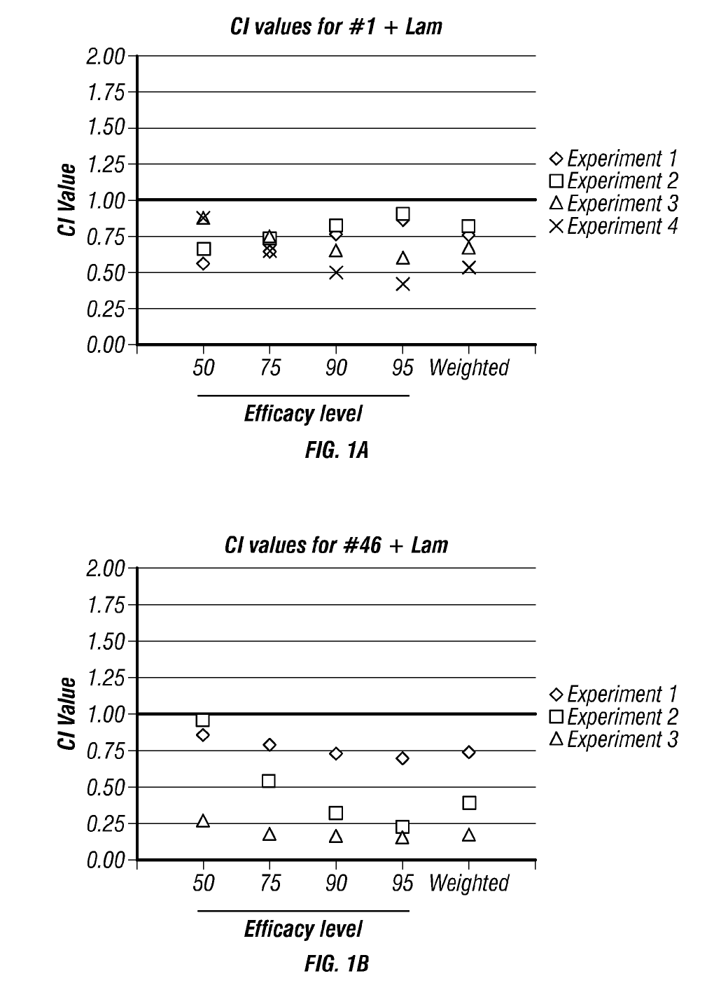

Current nucleos(t)ide analog therapy for HBV has converted hepatitis type B from an implacably progressing illness to a controllable disease. However, patients are only very rarely cured, in part due to the incomplete inhibition of HBV replication. The inventors hypothesized that the novel RNaseH inhibitors would work synergistically with the existing nucleos(t)ide analogs because the two classes of drugs target physically distinct active sites on the viral polymerase protein. Therefore, potential synergy between the RNAseH inhibitors and the nucleoside analog Lamivudine was analyzed using the Chou-Talalay method (Chou 2010). RNAseH inhibitors from two different chemical classes were employed, compound #1 [2-hydroxyisoquinoline-1,3(2H,4H)-dione], an HID, and #46 (β-thujaplicinol), an α-hydroxytropolone, were tested. Chou-Talaly analysis yields a combination index (CI). CI values <1.0 indicate synergy, CIs of approximately 1.0 indicate additive interactions, and CI values >1.0 indicate antagonism. CI values are calculated at various efficacy levels (EC₅₀, EC₇₅, EC₉₀, and EC₉₅), and a weighted CI value favoring higher efficacy levels is also generated. FIGS. 1A & 1B show the results of four experiments employing compound #1 and three with compound #46. All experiments revealed synergistic interactions between the RNAseH inhibitors and Lamivudine, and the weighted CI values were 0.70±0.1 for the HID compound #1 and 0.44±0.3 for the α-hydroxytropolone #46. Therefore, RNAseH inhibitors act strongly synergistically with an approved nucleos(t)ide analog drug against HBV. This demonstrates feasibility for employing RNAseH inhibitors in combination therapy with the nucleos(t)ide analogs during HBV treatment.

4. RNAseH Inhibitor Sensitivity is Insensitive to High Genetic Variation

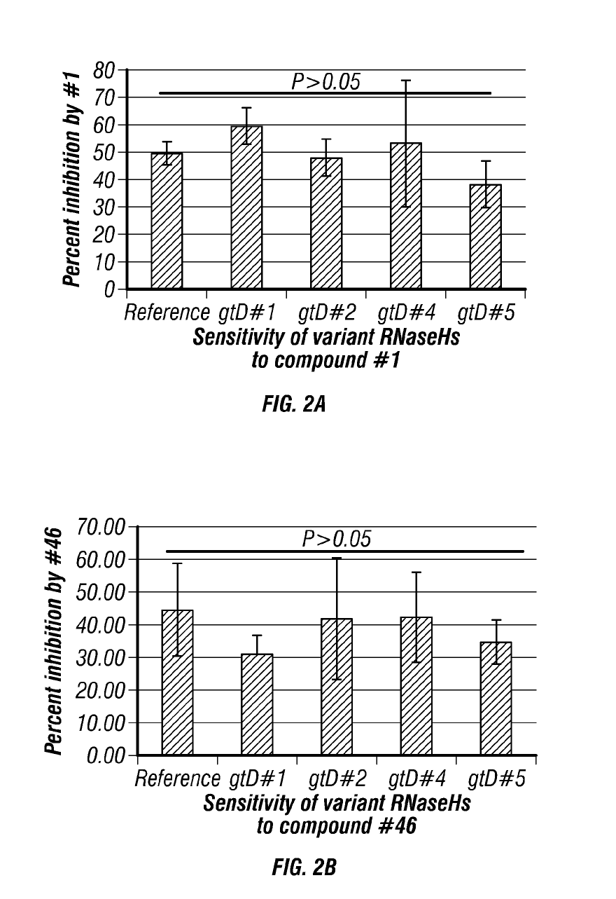

HBV has 8 genotypes differing in sequence by 8%. Genetic diversity in the RNAseH domain is about 6%, which is easily high enough to modulate viral sensitivity to RNAseH inhibitors. Therefore, the inventors tested the RNAseH inhibitors #1 [2-hydroxyisoquinoline-1,3(2H,4H)-dione], an HID, and #46 (β-thujaplicinol), an α-hydroxytropolone, for the ability to inhibit variant RNAseHs. Twelve purified, patient-derived RNAseH enzymes (4 from each genotypes B, C and D) were tested with the compounds at their respective IC₅₀ values in a biochemical RNAseH assay. FIGS. 2A & 2B demonstrate that the four genotype D enzymes each inhibited the HBV RNAseH by about 50% at the compounds IC₅₀ values as expected. Equivalent results were obtained for all 12 enzymes against compounds #1 and #46. Therefore, HBV's high genetic variation is unlikely to present a substantial barrier to drug development.

5. Results of Further Studies

A. Activity in the Oligonucleotide-Directed RNA Cleavage Assay

Effects on the activity of purified HBV RNaseH by compounds #1, 41-45, 78-91, 128, 138-140, and 190 (Table 3) was reported previously (Cai et al., 2014). The HBV RNaseH inhibition activity of the 16 previously untested compounds was examined using an oligonucleotide-directed cleavage assay. Compounds were incubated with the HBV RNaseH, a ³²P-labeled RNA substrate plus a complementary DNA oligonucleotide and compounds at 100, 60, and 20 μM and the amount of the RNA cleavage products was measured following resolution on an acrylamide gel. Of these 16 compounds, only compound #208 inhibited RNA cleavage by the HBV RNaseH.

TABLE 3
Activity of Compounds

| | | | | Inhibition of HBV | | | | | | | | |
|---|---|---|---|---|---|---|---|---|---|---|---|---|
| | | | | RNaseH | | | | | | | | |
| | | | | Oligo- | Molecular | HBV | | | | | | |
| | | | | nucleotide | Beacon | replication | HSV-1 | Inhibition | | | | |
| | | | | directed | cleavage | inhibition | suppression | of | Toxicity in | | | |
| Compound | Formal | Compound | Derivative | cleavage | assay | (EC₅₀, μm) | (log₁₀ at | huRNaseH | HepDES19 (g) | | | |
| # | Name | Structure | series (a) | assay (b, c) | (d) | (e) | 5 μM) (f) | 1 (b) | MTS | NR | LDH | TI |
| 208 | Sun B8155 | 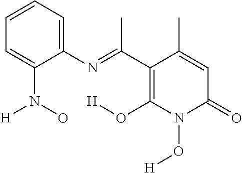 [SMILES: C/C(=N\c1ccccc1NO)c1c(C)cc(=O)n(O)c1O] | HPD | + | + | 0.69 ± 0.2 | − | − | 15 ± 7 | 13 ± 1 | 12 ± 0.1 | 22 |
| 211 | Rilopirox | 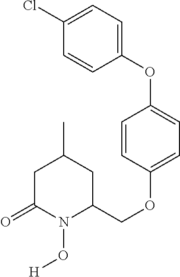 [SMILES: CC1CC(=O)N(O)C(COc2ccc(Oc3ccc(Cl)cc3)cc2)C1] | HPD | − | − | − | − | − | | | | |
| 86 | VS42 | 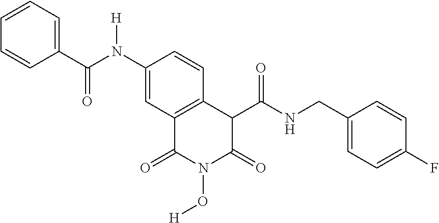 [SMILES: O=C(Nc1ccc2c(c1)C(=O)N(O)C(=O)C2C(=O)NCc1ccc(F)cc1)c1ccccc1] | HID | − | − | 1.4 ± 0.3 | − | + | 99 ± 2 | 52 ± 11 | >100 | 71 |
| 83 | MB88 | 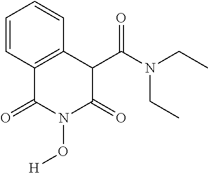 [SMILES: CCN(CC)C(=O)C1C(=O)N(O)C(=O)c2ccccc21] | HID | − | + | 2.3 ± 1.6 | − | + | 100 ± 35 | >100 | >100 | 43 |
| 88 | VS45 | 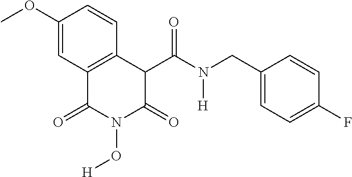 [SMILES: COc1ccc2c(c1)C(=O)N(O)C(=O)C2C(=O)NCc1ccc(F)cc1] | HID | − | − | 2.5 ± 0.5 | − | + | 40 ± 6 | 26 ± 2 | 57 ± 6 | 16 |
| 89 | VS51 | 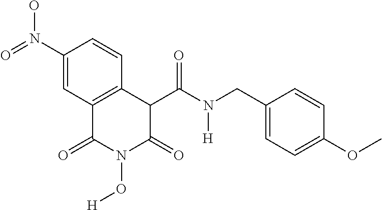 | HID | − | − | 2.6 ± 0.8 | − | + | 28 ± 8 | 19 ± 3 | 25 ± 4 | 11 |
| 1 | TRC 939800 | 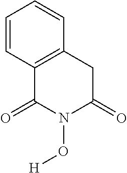 [SMILES: O=C1Cc2ccccc2C(=O)N1O] | HID | + | + | 4.2 ± 1.4 (b) | − | + | 75 ± 24 | 81 ± 4 | 79 ± 12 | 18 |
| 81 | MB4 | 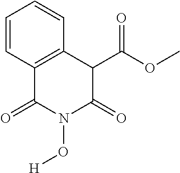 [SMILES: COC(=O)C1C(=O)N(O)C(=O)c2ccccc21] | HID | − | + | 4.4 ± 3.0 | − | + | 57 ± 16 | 31 ± 7 | 24 ± 5 | 13 |
| 91 | MB106 | 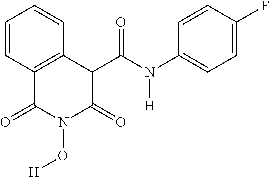 [SMILES: O=C(Nc1ccc(F)cc1)C1C(=O)N(O)C(=O)c2ccccc21] | HID | − | − | 4.6 ± 1.0 | − | + | 11 ± 4 | 33 ± 3 | 58 ± 22 | 2.4 |
| 87 | VS55 | 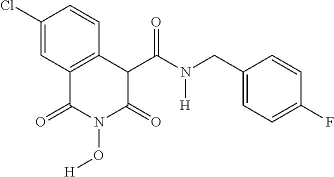 [SMILES: O=C(NCc1ccc(F)cc1)C1C(=O)N(O)C(=O)c2cc(Cl)ccc21] | HID | − | − | 4.5 ± 3.7 | − | + | 37 ± 17 | 24 ± 1 | 52 ± 15 | 8.2 |
| 85 | MB103 | 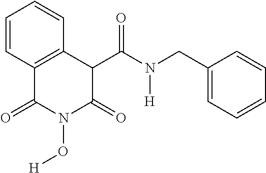 [SMILES: O=C(NCc1ccccc1)C1C(=O)N(O)C(=O)c2ccccc21] | HID | − | − | 6.5 ± 1.5 | − | + | 29 ± 12 | 67 ± 13 | >100 | 4.5 |
| 84 | MB105 | 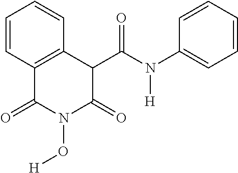 [SMILES: O=C(Nc1ccccc1)C1C(=O)N(O)C(=O)c2ccccc21] | HID | − | − | 6.5 ± 4.5 | − | + | 23 ± 9 | 32 ± 8 | 37 ± 15 | 3.5 |
| 90 | MB104 | 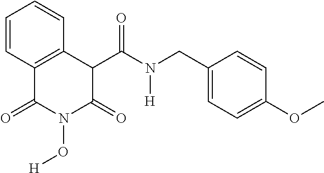 [SMILES: COc1ccc(CNC(=O)C2C(=O)N(O)C(=O)c3ccccc32)cc1] | HID | − | − | 19 ± 0.9 | − | + | 88 ± 17 | >100 | >100 | 4.6 |
| 82 | MB71 | 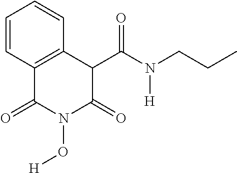 [SMILES: CCCNC(=O)C1C(=O)N(O)C(=O)c2ccccc21] | HID | − | | − | − | + | | | | |
| 78 | MB1 | 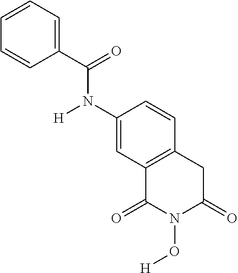 [SMILES: O=C(Nc1ccc2c(c1)C(=O)N(O)C(=O)C2)c1ccccc1] | HID | − | | − | − | + | | | | |
| 79 | MB2 | 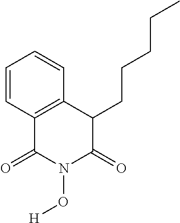 [SMILES: CCCCCC1C(=O)N(O)C(=O)c2ccccc21] | HID | − | | − | − | − | | | | |
| 80 | MB3 | 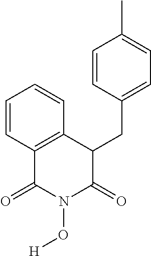 [SMILES: Cc1ccc(CC2C(=O)N(O)C(=O)c3ccccc32)cc1] | HID | − | | − | − | − | | | | |
| 41 | TRC C432800 (Ciclopirox) | 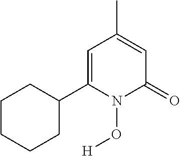 | POH | − | | − | 5.0 | + | | | | |
| 42 | Labotest 72543251 | 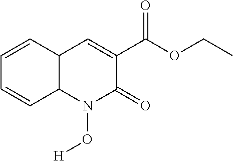 [SMILES: CCOC(=O)C1=CC2C=CC=CC2N(O)C1=O] | POH | − | | − | − | + | | | | |
| 43 | Sigma PH008969 | 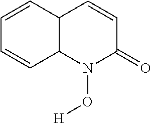 [SMILES: O=C1C=CC2C=CC=CC2N1O] | POH | − | | − | − | − | | | | |
| 44 | Labotest 12243782 | 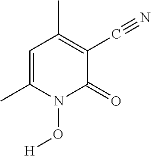 | POH | − | | − | − | − | | | | |
| 45 | TCI America H1040 | 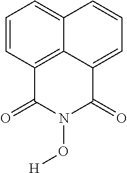 [SMILES: O=C1c2cccc3cccc(c23)C(=O)N1O] | HID | − | | − | − | − | | | | |
| 128 | Aldrichs elect CNC_ID 1006157 60 | 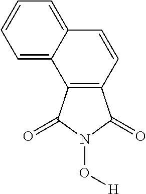 [SMILES: O=C1c2ccc3ccccc3c2C(=O)N1O] | POH | − | | | − | − | | | | |
| 132 | Aldrichs elect CNC_ID 3893067 67 | 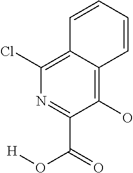 [SMILES: O=C(O)c1nc(Cl)c2ccccc2c1O] | POH | − | | − | | | | | | |
| 138 | Sigma H53704 | 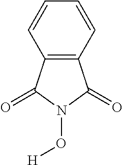 [SMILES: O=C1c2ccccc2C(=O)N1O] | POH | − | − | − | − | − | | | | |
| 139 | Sigma 130672 | 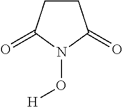 | POH | − | | − | | − | | | | |
| 140 | Chembridge 6325462 | 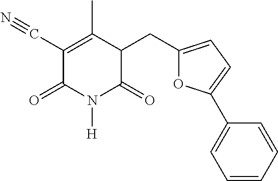 [SMILES: CC1=C(C#N)C(=O)NC(=O)C1Cc1ccc(-c2ccccc2)o1] | POH | − | | − | − | + | | | | |
| 191 | Piroctone olamine | 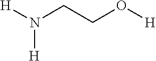 | POH | − | | − | 4.6 | − | | | | |
| | | 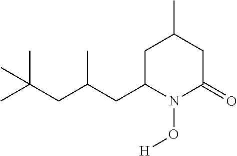 [SMILES: CC1CC(=O)N(O)C(CC(C)CC(C)(C)C)C1] | | | | | | | | | | |
| 197 | Benzoyle neurea | 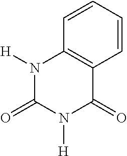 [SMILES: O=c1[nH]c(=O)c2ccccc2[nH]1] | POH | − | | − | − | − | | | | |
| 198 | 2,3- dihydroxy- quinoxaline | 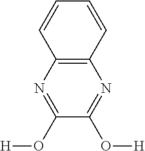 [SMILES: Oc1nc2ccccc2nc1O] | POH | − | | − | − | − | | | | |
| 204 | AK- 830/1321 7043 | 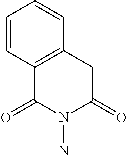 [SMILES: NN1C(=O)Cc2ccccc2C1=O] | POH | − | | − | − | + | | | | |
| 206 | AJ- 333/2500 6202 | 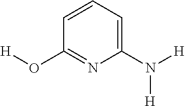 | POH | − | | − | − | + | | | | |
| 217 | Visas M Lab 4440351 42 | 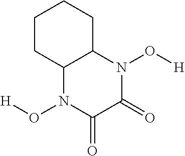 [SMILES: O=C1C(=O)N(O)C2CCCCC2N1O] | POH | − | | − | − | + | | | | |
| 236 | ZF4 | 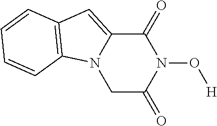 [SMILES: O=C1Cn2c(cc3ccccc32)C(=O)N1O] | FLT | − | | − | − | − | | | | |
| 237 | ZF13 | 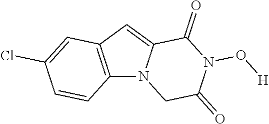 [SMILES: O=C1Cn2c(cc3cc(Cl)ccc32)C(=O)N1O] | FLT | − | | − | − | | | | | |
| 238 | ZF18 | 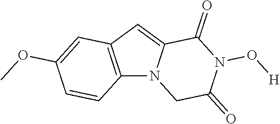 [SMILES: COc1ccc2c(c1)cc1n2CC(=O)N(O)C1=O] | FLT | − | | − | − | | | | | |
| 239 | ZF19 | 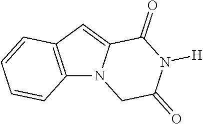 [SMILES: O=C1Cn2c(cc3ccccc32)C(=O)N1] | FLT | − | | − | − | | | | | |
| 240 | ZF24 | 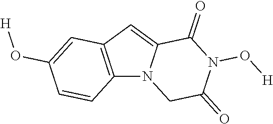 [SMILES: O=C1Cn2c(cc3cc(O)ccc32)C(=O)N1O] | FLT | − | | − | − | + | | | | |
| 241 | ZF29 | 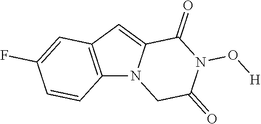 [SMILES: O=C1Cn2c(cc3cc(F)ccc32)C(=O)N1O] | FLT | − | | − | − | | | | | |

(a) N-hydroxyisoquinolinedione (HID), polyoxygenated heterocycles (POH), N-hydroxypyridinediones (HPD), and flutimides (FLT)
(b) Compounds 1-91 and 138-140 previously reported in (Cai et al., 2014)
(c) (+), Dose-dependent inhibition at ≤100 μM; (−), No inhibition detected at 100 μM
(d) (+), Dose-dependent inhibition at ≤500 μM; (−), No inhibition detected at 500 μM
(e) (−), No inhibition detected at 20 μM
(f) (−), ≤1 log₁₀ suppression of plaque forming units at 5 μM
(g) CellTiter 96 Aqueous Non-Radioactive Cell Proliferation Assay (MTS), neutral red retention assay (NR), lactate dehydrogenase release (LDH)

B. HBV Replication Inhibition and Cytotoxicity

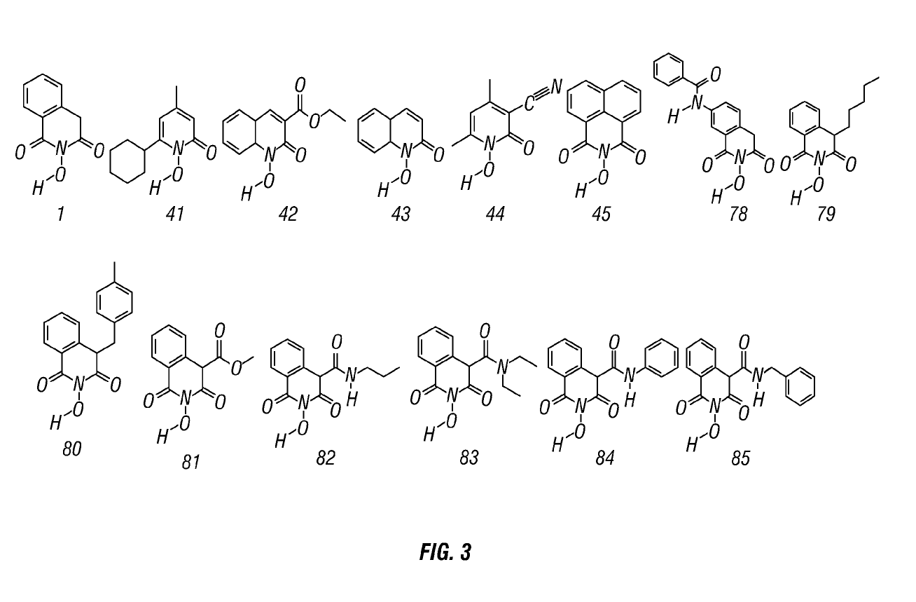

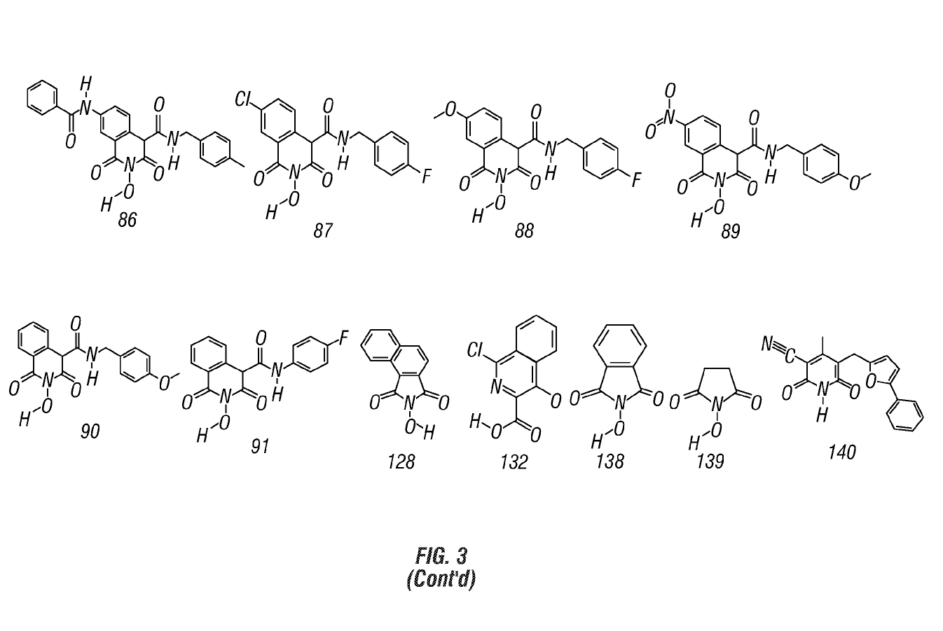

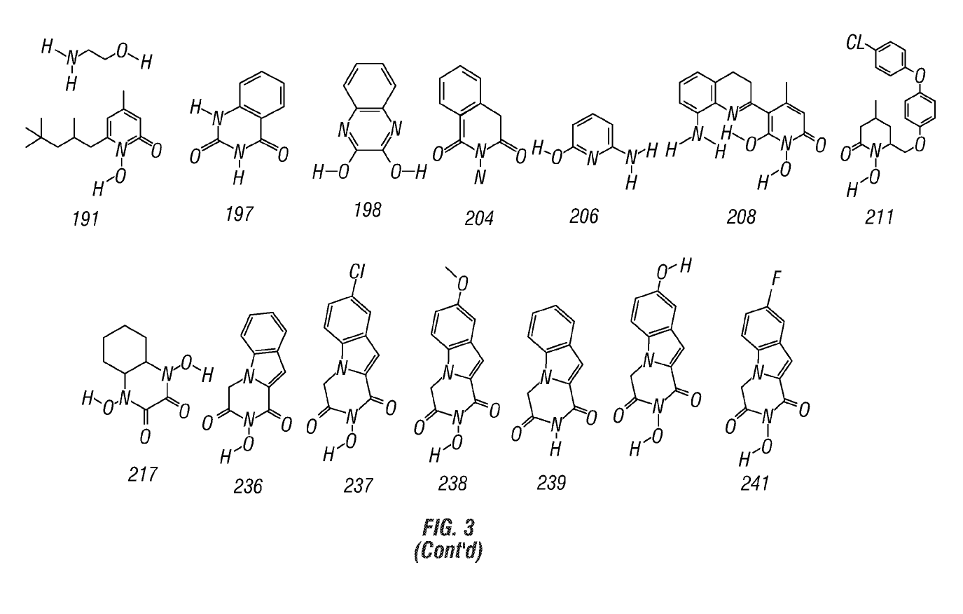

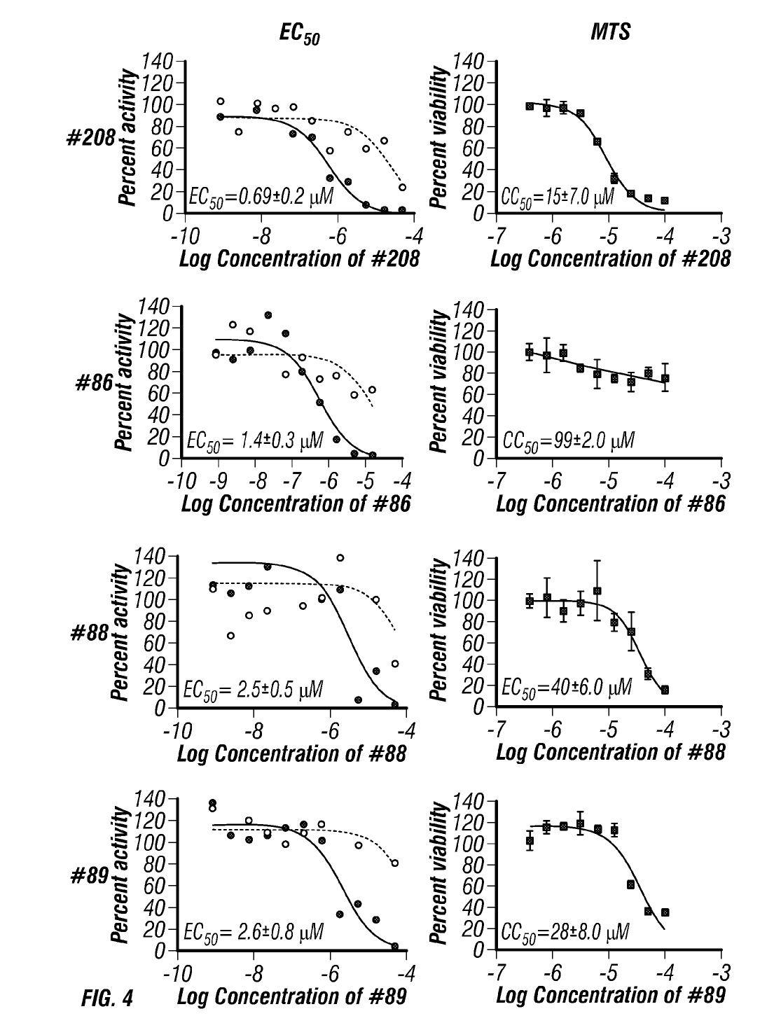

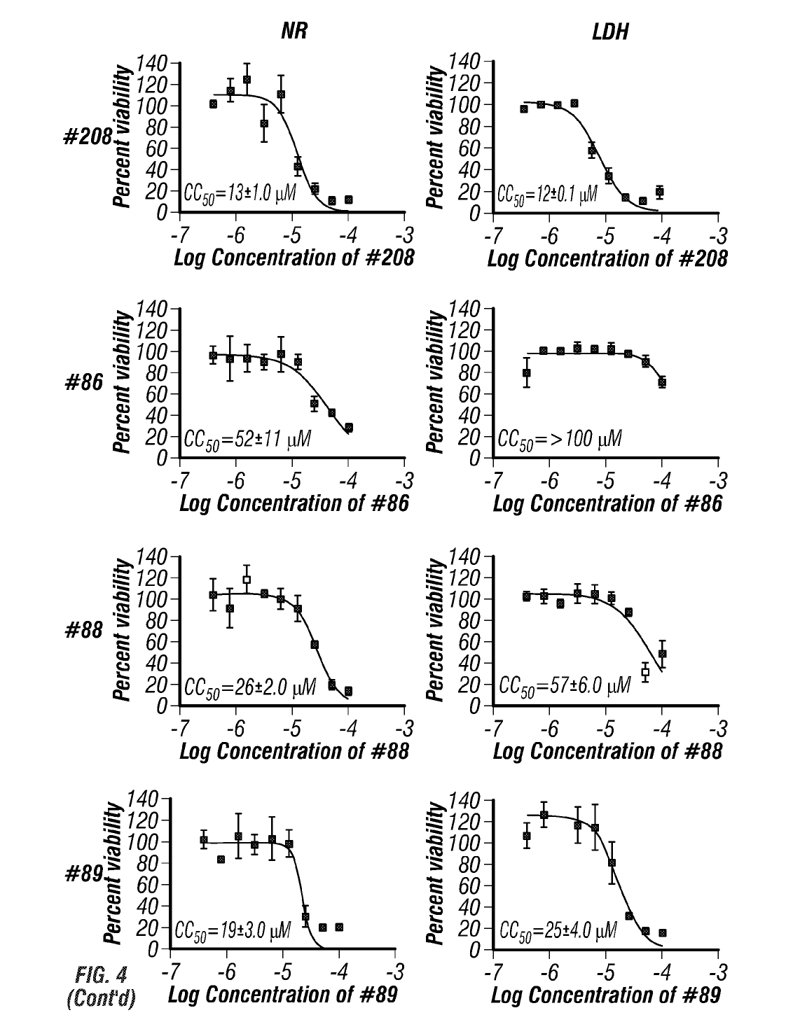

Thirty-nine HID, HPD, flutimide, and related POH compounds (Structures shown in FIG. 3) were screened for their ability to inhibit HBV replication in HepDES19 cells. HepDES19 cells are HepG2 cells that carry a stably transfected HBV genotype D genome under control of a tetracycline-repressible promoter (Guo et al., 2007). The cells were treated with the compound for three days and capsid-associated nucleic acids were quantified by strand-preferential quantitative PCR (q-PCR) because inhibition of the HBV RNaseH blocks positive-polarity DNA strand synthesis (Cai et al., 2014; Gerelsaikhan et al., 1996; Hu et al., 2013; Tavis et al., 2013). Inhibition was calculated as the amount of plus-polarity DNA relative to the DMSO treated controls. Qualitative screens at 20 μM identified 12 compounds that preferentially inhibited plus-polarity DNA synthesis, 11 HIDs and one HPD (Table 3). None of the flutimides or other POH compounds inhibited HBV replication. EC₅₀ values for suppression of both the plus- and minus-polarity DNA strands were then determined for all active compounds (Table 3 and representative data in FIG. 4). All of the active compounds preferentially inhibited positive-polarity DNA accumulation.

These preliminary hits were then screened for cytotoxicity using the MTS assay to evaluate the possibility that loss of HBV DNA in the q-PCR assay was due to cell death. CC₅₀ values were ranged from 11 to >100 μM (Table 3). EC₅₀ values were then calculated for the 12 hits by treating the cells replicating HBV with a wide range of compound concentrations. EC₅₀ values ranged from 0.69 to 18 μM (Table 1), with #208 having the lowest EC₅₀ value of 0.69 μM±0.2. FIG. 4 shows example CC₅₀ and EC₅₀ data for compounds #208, 86, 88, and 89. A therapeutic index (TI) (CC_{50(MTS)}/EC₅₀) comparing the CC₅₀ calculated by MTS assay and the EC₅₀ was determined for each compound; TI values ranged from 2.4 to 71.

To further evaluate potential cytotoxicity of these hits, the effect of the compounds was determined for the uptake of neutral red (NR) by the lysosomes and lactate dehydrogenase (LDH) release into the supernatant due to cell membrane permeability. The CC₅₀ values for the LDH assay ranged from 12 to >100 μM and the CC₅₀ values for the NR assay were 13 to >100 μM (Table 3). Overall, the TI values in Table 3 and FIG. 4 indicate that cytotoxicity was not the cause of antiviral efficacy, although it may have contributed to the apparent efficacy for #84, 85, 90, and 91, which all have TI values below 5.

C. Activity in an Improved RNaseH Assay

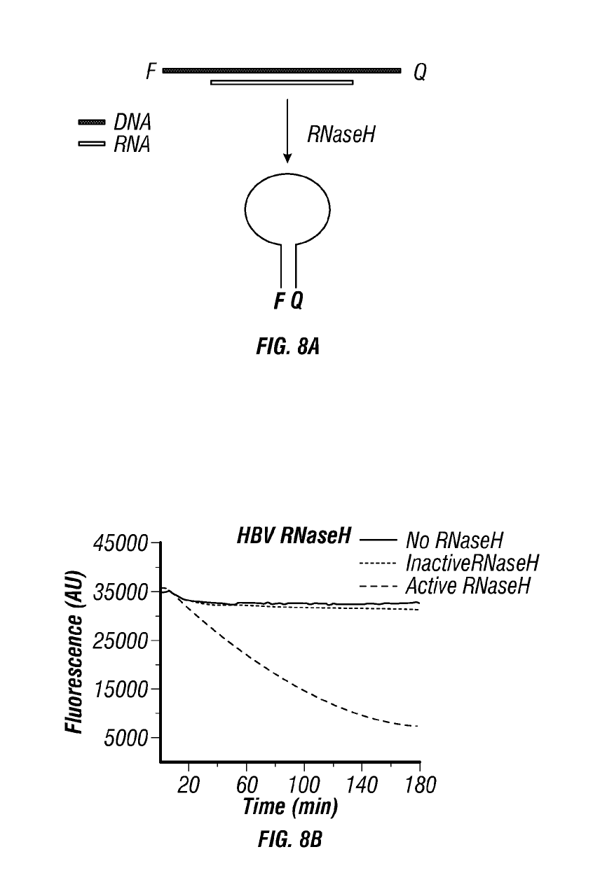

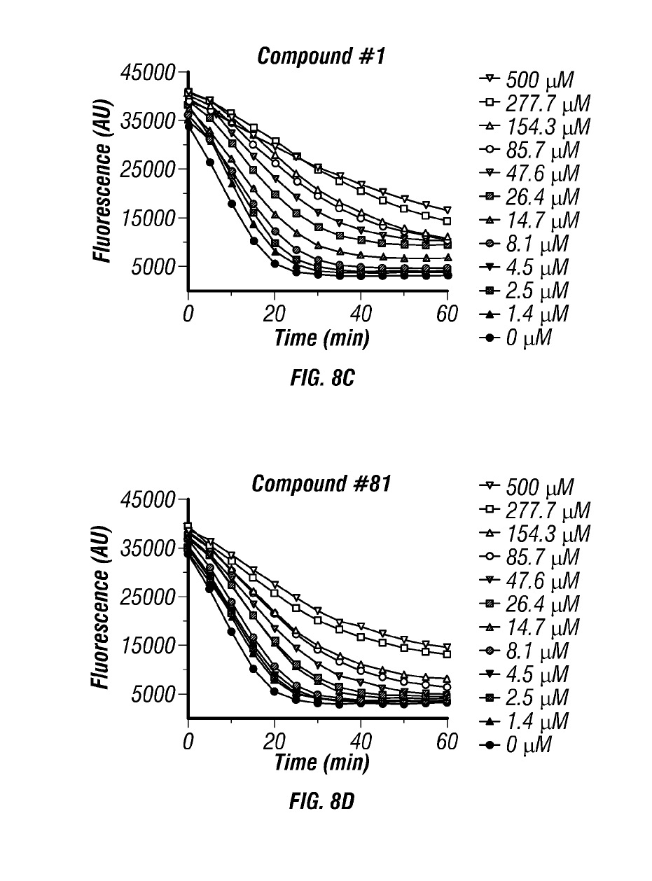

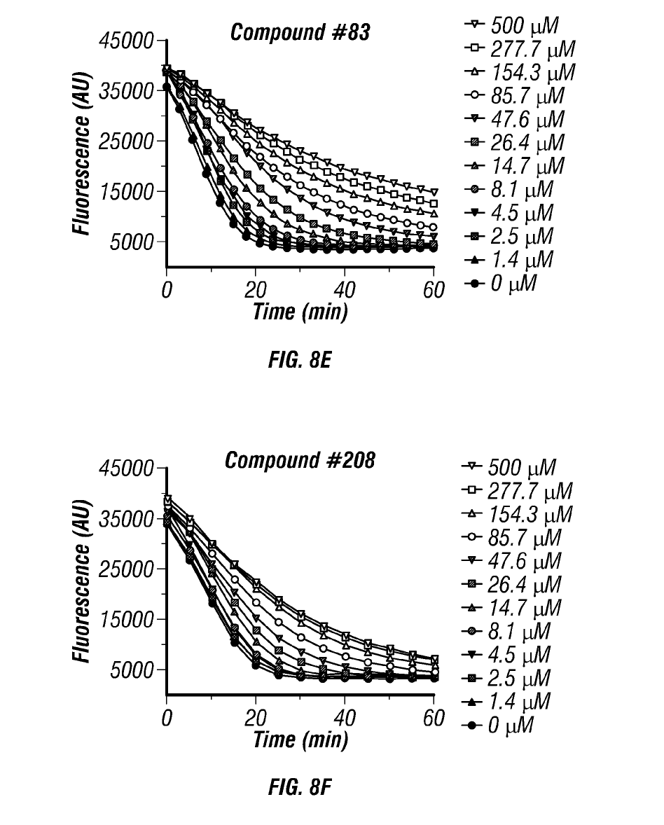

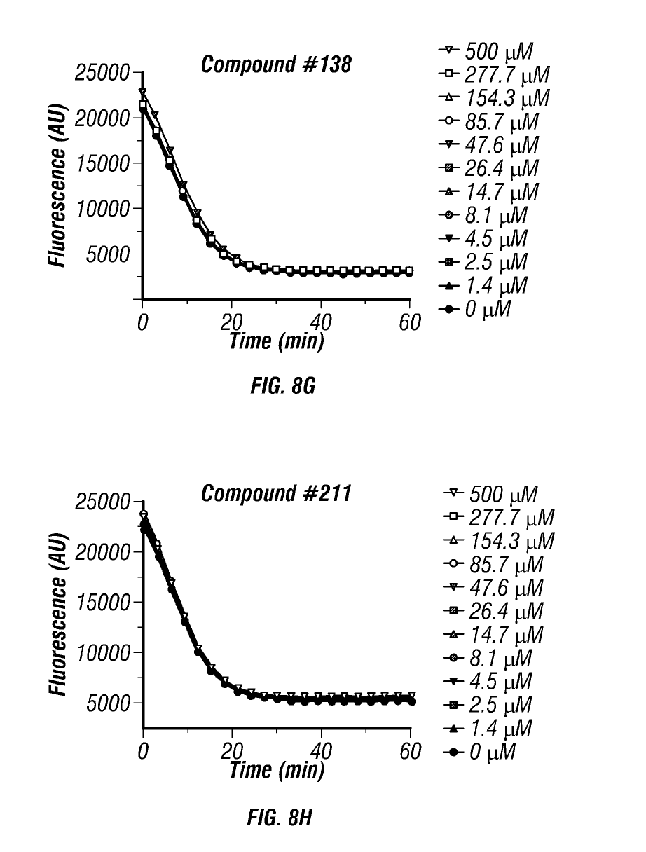

The lack of sensitivity in the oligonucleotide directed RNA cleavage assay led us to improve the purification of the recombinant RNaseH (Villa et al., 2016) and develop an alternative RNaseH assay that employs a molecular beacon (Chen et al., 2008). In this assay, a hairpin DNA oligonucleotide labeled with fluorescein on one end and a quencher on the other is held in a linear conformation by annealing to a complementary RNA; cleavage of the RNA causes the DNA to fold into a hairpin, suppressing fluorescence (FIG. 8A). Control reactions with 0.98 μg each the wild-type HBV RNaseH and an enzymatically inactive active site mutant (D702A/E731A) validated that loss in fluorescence in these reactions is due to action of the HBV RNaseH (FIG. 8B).

Compounds #1, 81, 83, and 208 inhibited reactions containing 2.1 μg purified HBV RNaseH in the molecular beacon assay, and negative control compounds #138 and #211 that cannot coordinate the active site Mg⁺⁺ ions had no effect on the rate of substrate degradation compared to the DMSO vehicle control (FIG. 8 and Table 3). This molecular beacon RNaseH assay is more sensitive than the oligonucleotide directed RNA cleavage assay at detecting HBV replication inhibitors. However, it still underestimates the degree of inhibition compared to activity seen in cells, and hence the assay at present is used only for qualitative interpretation.

D. Inhibition of the Human RNaseH1

Inhibition of purified huRNaseH1 by compounds #1, 41-45, 78-91, 138-140, and 190 (Table 3) was reported previously (Cai et al., 2014). We examined the huRNaseH1 inhibition activity of the previously untested compounds using an oligonucleotide-directed cleavage assay (Cai et al., 2014). Compounds were incubated with the huRNaseH1, a ³²P-labeled RNA substrate plus a complementary DNA oligonucleotide and compounds at 100, 60, and 20 μM. Compounds #204, 206, 217, and 240 detectably inhibited the huRNaseH1 (Table 1).

E. HSV-1 Replication Inhibition

Thirty-seven compounds were counter-screened for efficacy against HSV-1 to evaluate specificity of the inhibitors and to help determine if the efficacy against HBV replication may be due to an unsuspected indirect effect on the cells. Vero cells infected with HSV-1 at an MOI of 0.1 were treated with 5 μM of compound for 24 hours. Inhibition was calculated as log 10 suppression of plaque forming units compared with DMSO vehicle control. Of the tested compounds, only compounds #41 and 191 inhibited HSV-1 by more than 1 log 10.

F. Effect of the Compounds on HBV DNA Elongation

Compounds #1 (Cai et al., 2014), 81, 83, and 208 inhibited the HBV RNaseH in biochemical assays (Table 3). However, the other 8 HBV replication inhibitors did not suppress the HBV RNaseH in the biochemical assay, leading to questions about their mechanism of action. Anti-RNaseH activity is implied from their preferential suppression of the plus-polarity DNA strand (FIG. 4), but strand-preferential inhibition could also result from inhibiting the polymerase also. This is because positive-polarity DNA synthesis can be suppressed both directly by inhibiting the RNaseH enzyme and indirectly by reducing the amount of negative-polarity DNA strand that templates the positive-polarity strand, whereas minus-polarity DNA synthesis can only be suppressed by chain termination. To determine if the efficacy of these compounds was due to inhibition of the HBV polymerase, capsids isolated from HepDES19 cells were tested in the endogenous polymerase reaction (EPR) to measure DNA chain elongation by the HBV polymerase along the HBV nucleic acids within the capsids. Isolated HBV capsids were supplied with dATP, dTTP, dGTP, and [α³²P]dCTP in the presence of inhibitors at 4× their EC₅₀ values; negative control compounds #138 and #211 were tested at 30 μM. 1% DMSO was used as a vehicle control, and ddTTP (5.7 μM) was the positive control polymerase inhibitor. Samples were incubated at 37° C. for 7 hours to permit DNA chain elongation by the encapsidated HBV polymerase. Nucleic acids were purified, resolved on a 0.8% agarose gel and detected by autoradiography. DNAs from HBV capsids treated with the compounds incorporated ³²P similarly to DMSO control (FIG. 5). Minor variations were observed in signal intensities among the HID and HPD treated samples, but these variations were not consistent between multiple replicate experiments and none of them were statistically significant. In contrast, the chain terminator ddTTP strongly inhibited ³²P incorporation compared to the DMSO-treated samples in all assays (p≤0.001). Therefore, these HIDs and HPDs do not inhibit HBV DNA polymerization inside capsids, further implicating RNaseH inhibition as their mechanism of action.

E. Detection of Capsid-Associated RNA:DNA Heteroduplexes

To confirm that the strand preferential inhibition seen by the compounds was due to RNaseH inhibition, accumulation of RNA:DNA heteroduplexes in cells treated with the HID compound #89, which was inactive in both biochemical RNaseH assays, was detected by Southern blotting. Inhibiting the RNaseH blocks synthesis of the positive-polarity DNA strand by causing an accumulation of RNA:DNA heteroduplexes (Cai et al., 2014; Gerelsaikhan et al., 1996; Hu et al., 2013; Tavis et al., 2013). RNA:DNA heteroduplexes migrate as double-stranded products, but degradation of the RNA strand with exogenous RNase causes the DNAs to migrate as single-stranded species. To test for accumulation of RNA:DNA heteroduplexes, HepDES19 cells replicating HBV were treated with 4×EC₅₀ (10.4 μM) of compound #89 and 30 μM compound #138 (negative control). HBV cores were harvested after seven days of compound treatment. Capsid-associated nucleic acids were purified and split into two pools; one pool was treated with RNaseA and the other pool was mock treated. These nucleic acids were resolved by gel electrophoresis and detected by Southern analysis using a full-length double-stranded HBV ³²P-labeled probe.

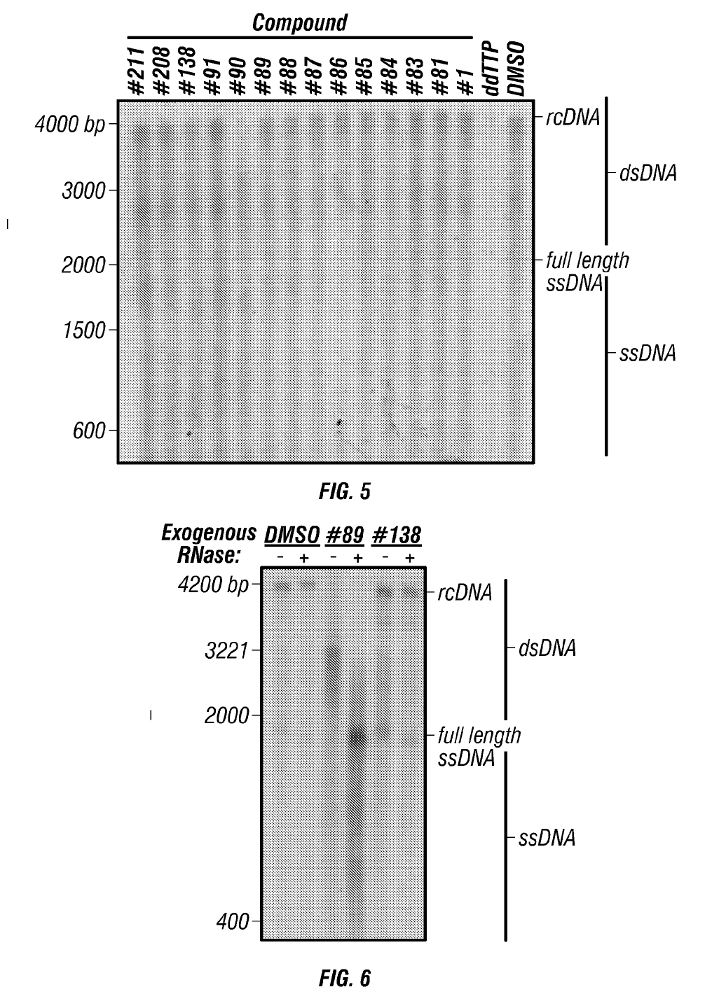

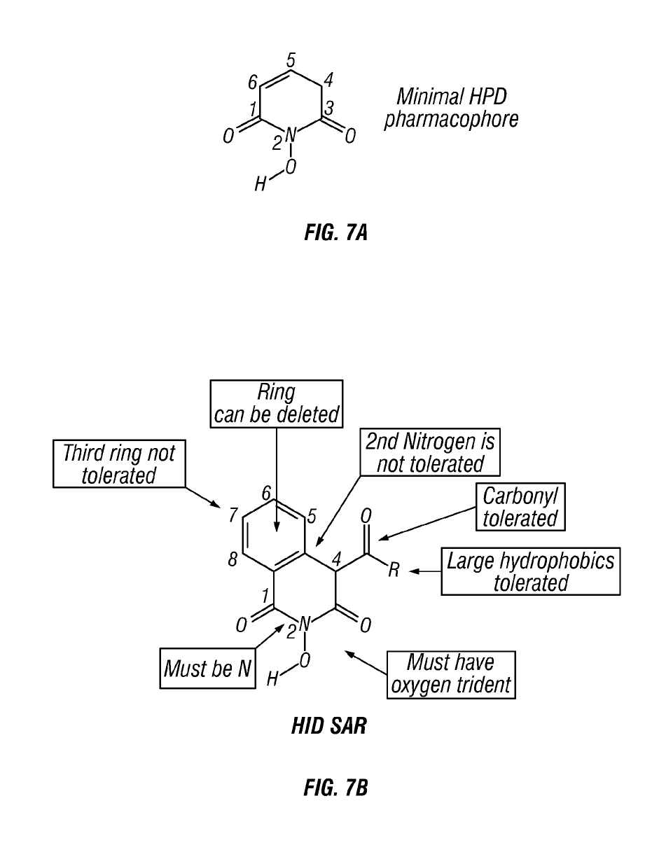

The DMSO vehicle-treated control revealed the expected accumulation of relaxed circular DNAs (rcDNA), shorter double-stranded species, and a smear of single-stranded products of varying lengths. Exogenous RNase treatment did not impact migration of these species (FIG. 6). Compound #138 was used as negative control as it does not inhibit HBV replication (Table 3), and migration of DNAs from cells treated with #138 was unaltered by exogenous RNase treatment. In contrast, treatment with 4×EC₅₀ of compound #89 caused the accumulation of truncated double-stranded species that migrated below the rcDNA in the DMSO control samples, plus accumulation of a smear of single-stranded species. Treatment with exogenous RNase to remove RNA from the sample resulted in faster-migrating single-stranded DNAs (FIG. 7). This indicates that compound #89 caused RNA:DNA heteroduplexes to accumulate by inhibiting the HBV RNaseH.

6. Discussion of Further Studies

It has been previously reported that compound #1, an HID, inhibits both the HBV RNaseH activity and HBV genomic replication (Cai et al., 2014). Based upon this initial study, it was concluded that the HID scaffold was not very promising for anti-HBV drug development because only one of 11 HIDs inhibited the HBV RNaseH in the oligonucleotide-directed RNA cleavage assay. However, further evaluation of this scaffold against HBV replication revealed a lack of sensitivity in the biochemical RNaseH assay for predicting inhibitors of HBV replication. Therefore, an additional 39 HIDs, HPDs, flutimides, and related POHs were screened for their ability to inhibit HBV replication. From this screen, 11 new inhibitors of HBV replication, 10 HIDs and one HPD, were identified with EC₅₀ values ranging from 0.69 to 19 μM and TI values of 2.4 to 71.

To improve sensitivity of the RNaseH assay, the recombinant RNaseH purification (Villa et al., 2016) was improved and developed an alternative assay that employs a molecular beacon (Chen et al., 2008). Using the molecular beacon assay, four compounds were identified as RNaseH inhibitors because they reduced the rate of substrate degradation with a dose-dependent pattern. Two of these inhibitors (#81 and 83) were previously reported as negative in the oligonucleotide-directed RNA cleavage assay. Although this newer RNaseH assay is more sensitive, it still under-reports the number of HBV replication inhibitors and is currently used only for qualitative assessment.

One possible explanation for the high false negative rate in ability of the biochemical assay to predict inhibitors of HBV replication is that the compounds may be metabolized to an active form upon entry to the cell. This is unlikely to be the only cause because compound #208 is stable in mouse liver microsomes. The compounds could become concentrated in cells, causing the increased efficacy in replication inhibition assays. However, this is also unlikely to be the only cause because the concentration would need to reach at least 50 to 100-fold higher in the cells to account for the discrepancy between the two assays. Lastly, the native conformation of the RNaseH protein is likely different inside the cell when compared to the recombinant enzyme used in the biochemical assay. The RNaseH is only one domain of the multifunctional viral polymerase, which exists as a larger protein complex associated with host chaperones (Hu and Seeger, 1996). In this biochemical assay, a purified fragment of the enzyme was used that contains only the RNaseH and lacks the chaperones. Without wishing to be bound by any theory, it is believe that the compounds have a higher affinity for the native enzyme complex than the recombinant protein and are therefore more active inside cells.

All compounds that inhibited HBV replication were screened in the MTS assay to measure mitochondrial function and had CC₅₀ values ranging from 11 to >100 μM (Table 3). To determine the breadth of cytotoxicity of these hits, the cytotoxicity screens were expanded to include lysosomal function using the neutral red release assay and cell membrane permeability using the LDH release assay. Those compounds with low CC₅₀ values in the MTS assay also had significant cytotoxicity in the neutral red and LDH release assays, and compounds with little evidence of cytotoxicity in the MTS assay were also fairly nontoxic in the other two assays (Table 3 and FIG. 4). The concordance among the assays in this initial cytotoxicity panel implies that these assays may all be measuring secondary cytotoxic effects on the cells rather than primary cytotoxic events. Regardless, a good estimation of the compounds' adverse cellular effects can be obtained from the MTS assay alone.

Specificity of these compounds was evaluated by counter-screening for activity against the huRNaseH1. The huRNaseH1 is a potential off-target host enzyme for these compounds, and inhibition of the huRNaseH1 could cause significant toxicity during the long-term drug exposure likely to be required for curative anti-HBV therapy. All 11 HIDs inhibited the huRNaseH1 in the qualitative screens at 20 μM (Cai et al., 2014). However, the HPD, compound #208, did not inhibit the huRNaseH1 at 100 μM (Table 3). These data demonstrate a need to expand selectivity for the HBV RNaseH over the huRNaseH1, but the high degree of selectivity of #208 indicates that improved selectivity is possible.

The compounds were also counter-screened 37 compounds for efficacy against HSV-1. None of the 12 inhibitors of HBV replication inhibited HSV replication by more than 1 log₁₀ at 5 μM (Table 3). Only compounds #41 and 191, which are both negative against HBV replication, inhibited HSV-1 by >3 log₁₀. Previous suppression levels observed for strong HSV-1 inhibitors in this assay were >5 log₁₀ at 5 μM (Tavis et al., 2014). This implies that there is little to no cross-reactivity of the compounds with HSV-1, and consequently increases our confidence that these compounds are not working by some unsuspected indirect effect on the cell.

HIDs have been tested extensively for activity against the HIV RNaseH and were found to be potent inhibitors in vitro against the purified enzyme (Billamboz et al., 2008; Billamboz et al., 2011a; Billamboz et al., 2011b; Billamboz et al., 2016; Billamboz et al., 2013; Desimmie et al., 2013; Hang et al., 2004; Klumpp et al., 2003; Suchaud et al., 2014). However, efficacy of these compounds against HIV replication is significantly lower (Billamboz et al., 2008; Billamboz et al., 2011a; Billamboz et al., 2011b; Billamboz et al., 2016; Billamboz et al., 2013; Chung et al., 2011; Fuji et al., 2009; Himmel et al., 2009; Kirschberg et al., 2009; Su et al., 2010; Suchaud et al., 2014). In comparison, the HIDs inhibited the HBV RNaseH in vitro poorly, but several HIDs inhibited HBV replication well in culture (Table 3). Compound #81 inhibits HIV replication with an EC₅₀ of 13.4 μM (Billamboz et al., 2011b) and HBV replication at 4.4 μM, a 3-fold improvement. Compound #86 inhibits HIV replication at >21 μM (Suchaud et al., 2014) but inhibited HBV replication at 1.4 μM, a 15-fold improvement. In general, inhibition of HBV replication by HIDs is more potent than that of HIV replication despite the HIDs being better inhibitors of HIV RNaseH in the biochemical assays. The ability of the HIDs to inhibit both HIV and HBV indicates they may be useful for the treatment of both HIV and HBV. However, it is too early to determine if development of a dual specificity inhibitor such as Tenofovir may be feasible.

The primary screening results and EC₅₀ curves show strong preferential inhibition of the plus-polarity DNA strand (FIG. 4), which is a hallmark of HBV RNaseH inhibition (Hu et al., 2013; Tavis et al., 2013). However, this strand preferentiality could also result from inhibition of DNA elongation by the HBV polymerase because positive-polarity DNA synthesis can be suppressed both directly by inhibiting the enzyme and indirectly by reducing the amount of negative-polarity DNA strand that templates the positive-polarity strand. To rule out the possibility that observed strand preferentiality is caused by inhibition of the HBV polymerase rather than RNaseH inhibition, DNA elongation by the native HBV polymerase within viral capsids was measured (FIG. 5). The positive control, the DNA chain terminator ddTTP, reduced DNA elongation by ˜90% (p<0.001). None of the HBV inhibitors or the two negative control compounds (#138 and 211) significantly altered incorporation of [α³²P]dCTP into the growing DNA chains. Therefore, the strand preferentiality seen in replication inhibition assays with the HID and HPD compounds is not a result of inhibiting the HBV polymerase. To confirm that an HID inhibitors can target the HBV RNaseH, we characterized the effects that compound #89 had on HBV DNAs in capsids. Despite being negative in the biochemical assay (Table 3), compound #89 prevented formation of rcDNA and caused the accumulation of RNA:DNA heteroduplexes (FIG. 6), demonstrating that it is an RNaseH inhibitor. Together, inhibition of purified HBV RNaseH by compounds #1, 81, 83, and 208 (Table 3), the preferential inhibition of the positive-polarity DNA strand by all active compounds (FIG. 4), the failure of all active compounds to inhibit elongation of DNAs by the HBV polymerase within capsids (FIG. 5), and the accumulation of RNA:DNA heteroduplexes in cultures treated with #89 indicates that the HID and HPD compounds inhibit HBV replication primarily by suppressing the viral RNaseH.

These data permit the generation of a preliminary structure activity relationship (SAR) with five key features (FIG. 7). The six-membered nitrogenous HPD ring appears to be the minimal pharmacophore because it is shared by all active compounds. Addition of a second six-membered ring bridging positions five and six of the HPD ring creates the HID scaffold. Both the HPD and HID scaffolds have an oxygen trident at positions R¹-R³ which is essential for their activity. The loss of any one of these oxygens results in inactive compounds. For example, compound #204 is lacking the oxygen at R² and is inactive. Compound #211 (HPD) lacks the oxygen at R³ and is inactive (FIG. 7B). This is consistent with the known mechanism by which the HIDs inhibit the HIV RNaseH (Billamboz et al., 2008; Billamboz et al., 2011a; Billamboz et al., 2011b; Billamboz et al., 2016; Billamboz et al., 2013; Desimmie et al., 2013; Hang et al., 2004; Klumpp et al., 2003; Suchaud et al., 2014). Compounds that contain a five-membered ring (#138 and #139) are also inactive, presumably due to an inability to coordinate the Mg⁺⁺ ions at the active site of the HBV RNaseH. This failure could be due to inappropriate bond angles and/or to the lack of a relatively acidic hydrogen at R⁴ of the HID ring. Substitutions by alkyl groups at R⁴ (compounds #79 and #80) are not tolerated, whereas including a carbonyl group (ester or amide functions) at R⁴, which increases the acidity of the hydrogen linked to carbon 4 and therefore the ability to coordinate the Mg⁺⁺ ions (Billamboz et al., 2011b), followed by large hydrophobic groups (aryl or alkaryl) is tolerated. Lastly the addition of a third ring condensed in positions R⁶ and R⁷ and a second nitrogen on the HID scaffold to create the flutimides is not tolerated (FIG. 7B). Six flutimides carrying this third ring and second nitrogen were tested and none of them were active against HBV replication or the HBV RNaseH. This preliminary SAR is being used to guide a hit-to-lead medicinal chemistry campaign.

7. Methods and Materials of Further Studies

A. Compound Acquisition and Synthesis

Compound structures and chemical names are in FIG. 3 and Table 3. Compounds #1, 41-45, 78-91, 138-140, and 190 were reported previously (Cai et al., 2014). Compounds #236-241 were synthesized as previously reported (Zoidis et al., 2016). Compounds #128, 132, 191, 197, 198, 204, 206, 208, 211, and 217 were purchased commercially. All compounds used in this study were ≥95% pure. The compounds were dissolved at 10 mM in DMSO and stored in opaque tubes at −80° C.

B. Expression and Purification of HBV RNaseH and huRNaseH1

HuRNaseH1 and HBV RNaseH were expressed in Escherichia coli and purified by nickel-affinity chromatography as described (Villa et al., 2016).

C. RNaseH Assays

The oligonucleotide-directed RNA cleavage assay was reported previously (Hu et al., 2013; Tavis et al., 2013). Briefly, a ³²P-labeled RNA was combined with a DNA oligonucleotide and the RNA:DNA substrate was incubated in the presence of the RNaseH and test compounds in 50 mM tris pH 8.0, 190 mM NaCl, 5 mM MgCl₂, 3.5 mM DTT, 0.05% NP40, 6% glycerol, and 1% DMSO at 42° C. for 90 minutes. The products were resolved by gel electrophoresis and detected by audioradioagraphy. 50% inhibitory concentration (IC₅₀) values were calculated with GraphPad Prism using the log [inhibitor] vs. response three parameter algorithm. Inhibition was qualitatively determined as a dose-dependent reduction in the amount of substrate degraded in the reaction.

Inhibition of HBV RNaseH was also evaluated using a molecular beacon fluorescence assay originally developed for the HIV enzyme (Chen et al., 2008). Purified HBV RNaseH (2.1 μg) was added to RNaseH buffer (50 mM HEPES pH 8.0, NaCl 100 mM, TCEP 2 mM, Tween 20 0.05%), an DNA/RNA heteroduplex substrate (25 nM), and 20 units of RNaseOut in the presence of 0 to 500 μM of the inhibitors in a final concentration of 5% DMSO in a 100 μL reaction. The substrate is a hairpin DNA oligonucleotide with a 5′ fluorescein reporter and a 3′ black hole quencher annealed to a complementary RNA oligonucleotide. The reaction was initiated by adding 5 mM Mg⁺⁺, and fluorescence was monitored at 37° C. with a Synergy 4 96-well plate reader. Inhibition was qualitatively determined as a dose-dependent reduction in the rate of substrate degradation.

D. Cells and Cell Culture

HepDES19 cells were maintained in Dulbecco's modified Eagle's medium (DMEM)/F12 media supplemented with 10% fetal bovine serum (FBS) and 1% penicillin/streptomycin (P/S) with 1 μg/mL tetracycline. Tetracycline was removed to induce expression of HBV. Test compounds were applied to the cells in the presence of 1% DMSO.

Vero cells were maintained DMEM supplemented with 3% newborn calf serum, 3% bovine growth serum, 2 mM L-glutamine, and P/S. Test compounds were applied to the cells in the presence of 0.05% DMSO. The herpes simplex virus 1 (HSV-1) strain used for screening was a de-identified clinical isolate from the Saint Louis University Hospital passaged once in culture. Virus titers were determined as previously described (Knipe and Spang, 1982; Morrison and Knipe, 1996).

E. HBV Replication Inhibition Assay

HBV replication inhibition was determined using HepDES19 cells as previously described (Cai et al., 2014). Briefly, HepDES19 were seeded in 12-well plates at 2×10⁵ cells per well in the absence of tetracycline. Test compound was applied to cells 48 hours after removal of tetracycline. Cells were lysed 3 days after compound addition, and nonencapsidated nucleic acids were digested with micrococcal nuclease as described (Hu et al., 2013). HBV DNA was purified from capsids using a QIAamp pathogen minikit with proteinase K digestion extended to overnight at 37° C. TaqMan PCR was performed for 40 cycles with an annealing temperature of 60° C. The primers and probe (IDT Inc.) for the plus-polarity DNA strand were 5′CATGAACAAGAGATGATTAGGCAGAG3′, 5′GGAGGCTGTAGGCATAAATTGG3′, and 5′/56-FAM/CTGCGCACC/ZEN/AGCACCATGCA/3IABkFQ. The primers and probe for the minus-polarity DNA strand were 5′GCAGATGAGAAGGCACAGA3′, 5′CTTCTCCGTCTGCCGTT3′, and 5′/56-FAM/AGTCCGCGT/ZEN/AAAGAGAGGTGCG/3IABkFQ. The effective concentration 50% (EC₅₀) values were calculated with GraphPad Prism using the four-parameter log(inhibitor)-versus-response algorithm with the bottom value set to zero.

F. HSV-1 Replication Inhibition Assay

Vero cells were plated in 24-well plates and infected with HSV-1 at a multiplicity of infection (MOI) of 0.1 as previously described (Tavis et al., 2014). Compounds and virus were diluted in phosphate buffered saline (PBS) containing 2% newborn calf serum and 2 mM L-glutamine so that the final concentration of compound was 5 μM. The cells were incubated at 37° C. for 1 hour with the virus containing inoculum, then the inoculum was removed and the wells were washed once in PBS. Compound diluted to 5 μM in supplemented DMEM was added and cells were incubated at 37° C. for an additional 23 hours. The plates were then microscopically inspected for cytopathic effect (CPE) or toxicity and then frozen at −80° C. Virus titers for wells with limited CPE compared to DMSO vehicle-treated controls were then determined by plaque assay on Vero cells. Each experiment was repeated at least once.

G. Cytotoxicity Assays

HepDES19 cells were seeded at 1×10⁴ cells per well in a 96 well plate in the absence of tetracycline. The test compounds were applied in triplicate to the cells 48 hours later and the cells were incubated for 72 hours. Cell viability was measured using the CellTiter 96 Aqueous Non-Radioactive Cell Proliferation Assay (Promega). [3-(4,5-dimethylthiazol-2-yl)-5-(3-carboxymethoxyphenyl)-2-(4-sulfophenyl)-2H-tetrazolium, inner salt] (MTS) and phenazine methosulfate (PMS) solution at 2 mg/mL MTS and 0.043 mg/mL PMS were added to the medium, the cells were incubated for two hours at 37° C., and absorbance was measured at 490 nm. Neutral red uptake was measured as described (Repetto et al., 2008). Neutral red was added to the media at a final concentration of 40 μg/mL. Cells were washed with PBS and incubated with neutral red medium for two hours at 37° C. The neutral red medium was removed and the cells were washed with PBS. Neutral red destain solution (50% ethanol, 49% deionized water, 1% glacial acetic acid) was added, the plate was shaken rapidly for 10 minutes, and absorbance was measured at 540 nm. Lactate dehydrogenase (LDH) release was measured using the LDH-cytotoxicity colorimetric assay kit (BioVision). Cells were lysed with 1% Triton-X to provide the high control and DMSO vehicle treatment was the low control. Supernatants were collected and transferred to a fresh 96 well plate, and LDH reaction mixture containing the catalyst and dye solution was added. The reaction was incubated for 15-20 minutes at room temperature in a dark box and absorbance was measured at 490 nm. Percent cytotoxicity was calculated as [(test sample−low control)/(high control×low control)]×100. The cytotoxic concentration 50% (CC₅₀) values were calculated with GraphPad Prism by using the four-parameter variable-response log(inhibitor)-versus-response algorithm with the bottom value set to zero.

H. Core Particle Isolation and Endogenous Polymerase Reaction

HBV cores were harvested from HepDES19 cells grown in the absence of tetracycline for 20 days using polyethylene glycol 8000 precipitation (Guo et al., 2003). Cells were rinsed in PBS and lysed in 1 mL core lysis buffer with 2 μL of protease inhibitor cocktail (Sigma) at room temperature for 10 minutes. Cell lysates were collected and 10 mM CaCl₂) was added. The lysate was centrifuged for 5 minutes at 21,000 g, the supernatant was incubated with 150 U of micrococcal nuclease at 37° C. for 60 minutes, and then 27 mM EDTA was added to stop the reaction. 333 μL of 26% PEG8000 was added to precipitate core particles and incubated at 4° C. for 6 hours. The samples were centrifuged at 21,000 g for 20 minutes at 4° C. The supernatant was removed and the pellet was resuspended in 250 μL of 10 mM Tris buffer pH 7.5. Cores were stored at −80° C.

The endogenous polymerase reaction was done using a modified procedure from (Nguyen et al., 2007). 50 μL of cores were incubated with 4 mM CaCl₂) and 15 U of micrococcal nuclease at 37° C. for 30 minutes. The reaction was terminated with 5.7 mM EGTA. The EPR reaction contained 50 mM Tris-HCl, pH 7.5, 10 mM MgCl₂, 0.1% NP-40, 0.1% β-mercaptoethanol, 150 mM NaCl, 5.7 μM each dTTP, dGTP, dATP, 1 μL of [α³²P]dCTP (10μ Ci) and test compounds at 4×EC₅₀ or 1% DMSO as a vehicle control. The reaction was incubated at 37° C. for 7 hours and terminated by adding EDTA to 10 mM. Nucleic acids were purified by incubating the cores overnight with proteinase K (0.5 mg/mL) and 1.5% SDS at 37° C., followed by phenol-chloroform extraction and ethanol precipitation. These nucleic acids were dissolved in tris-EDTA buffer, resolved on a 0.8% agarose gel, transferred to a HyBondN membrane, and detected by autoradiography. Band intensities for each experiment were quantified using Image J and percent inhibition was calculated relative to the DMSO-treated reactions. Statistical significance of variation in band intensities was evaluated using a one-way ANOVA with a Dunnett post hoc analysis, with p=0.05 indicating significance.

I. Detection of Capsid-Associated RNA:DNA Heteroduplexes

HepDES19 cells were plated in 100 mm dishes without tetracycline and incubated for 72 hours in the absence of tetracycline before addition of compound #89 at 4×EC₅₀ (10.4 μM) and the negative control compound #138 at 30 μM; compound-containing media was replaced every three days. HBV nucleic acids were purified seven days after compound addition. HBV core particles were isolated by sedimentation through a sucrose cushion as described (Tavis et al., 1998). Cores were treated overnight with proteinase K (0.5 mg/mL) and 1.5% SDS at 37° C. Nucleic acids were purified by phenol-chloroform extraction and ethanol precipitation. The nucleic acids were split into two pools. One pool was treated with RNaseA (1.5 μg/μL) at 37° C. for 30 minutes and the other was mock-treated. The nucleic acids were resolved by electrophoresis on a 0.8% agarose gel and detected by Southern analysis using a ³²P-labeled full-length HBV DNA probe to detect both the plus-polarity and minus-polarity HBV DNA strands by autoradiography or phosphorimage analysis.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference:
Ariyoshi et al., Cell 78:1063-1072, 1994.
Bokesch et al., J. Nat. Prod. 71, 1634-1636, 2008.
Budihas et al., Nucleic Acids Res. 33, 1249-1256, 2005.
Cai, et al., Antiviral Res., 108:48-55, 2014.
Chou, et al., Cancer Res., 70:440-446, 2010.
Chung et al., Antimicrob. Agents Chemother. 54, 3913-3921, 2010.
Didierjean et al., Antimicrob. Agents Chemother. 49, 4884-4894, 2005.
Dyda et al., Science 266:1981-1986, 1994.
Frank et al., Biol. Chem. 379:1407-1412, 1998.
Frank et al., Proc. Natl. Acad. Sci. USA 95:12872-12877, 1998.
Himmel et al., ACS Chem. Biol. 1:702-712, 2006.
Hostomsky et al., Nulceases, vol. 2, 1993b.
Hostomsky et al., “Ribonuclease H,” in: Linn, S. M., Lloyd, R. S., Roberts, R. J. (Eds.), Nulceases. Cold Spring Harbor Laboratory Press, Plainview, N.Y., pp. 341-376, 1993a.
Hostomsky et al., Structure 3:131-134, 1993c.
Hu et al., Antiviral Res., 99:221-229, 2013.
Katayanagi et al., Nature 347: 306-309, 1990.
Keck et al., J. Biol. Chem. 273, 34128-34133, 1998.
Klarmann et al., AIDS Rev 4: 183-194, 2002.
Klumpp et al., Nucleic Acids Res. 31, 6852-6859, 2004.
Klumpp and Mirzadegan, Curr. Pharm. Des 12:1909-1922, 2006.
Kwun et al., J. Gen. Virol. 82, 2235-2241, 2001.
Lai et al., Structure 8:897-904, 2000.
Li et al., Mol. Biol. Evol. 12:657-670, 1999.
Lima et al., Methods Enzymol. 341:430-440, 2001.
Lu, et al., Antimicrob. Agents Chemother., 59(2):1070-1079, 2015.
Nowotny et al., Cell 121: 1005-1016, 2005.
Nowotny, EMBO Rep. 10:144-151, 2009.
Parker et al., EMBO J. 23: 4727-4737, 2004.
Reyes et al., Arch Intern Med, 163:76-80, 2003.
Shaw-Reid et al., J. Biol. Chem. 278, 2777-2780, 2003.
Smith et al., J. Virol. 68, 5721-5729, 1994.
Song et al., Science 305: 1434-1437, 2004.
Su et al., J. Virol. 84:7625-7633, 2010.
Takada et al., J. Nat. Prod. 70, 1647-1649, 2007.
Tavis and Lomonosova, Antiviral Res., 118:132-138, 2015.
Tavis et al., PLoS pathogens 9:e1003125, 2013.
Wendeler et al., ACS Chem. Biol. 3, 635-644, 2008.
Williams et al., Bioorg. Med. Chem. Lett. 20:6754-6757, 2010.
Yang et al., Science 249: 1398-1405, 1990.
Yang and Steitz, Structure, 3, 131-134, 1995.

What is claimed:

1. A method of inhibiting hepatitis B virus replication comprising contacting the hepatitis B virus with an effective amount of a compound of the formula: 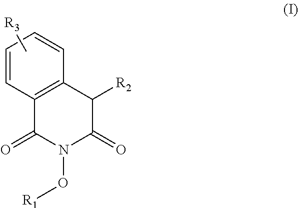
wherein:
R₁ is hydrogen or alkyl_{(C≤8)}, acyl_{(C≤8)}, or a substituted version of either of these groups;
R₂ is alkyl_{(C≤12)}, aryl_{(C≤12)}, aralkyl_{(C≤12)}, or a substituted version of any of these groups; or —C(O)ORₐ or —C(O)NRₐR_{b}, wherein:
Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
or a pharmaceutically acceptable salt thereof.

2. The method of claim 1, wherein the compound is further defined as: 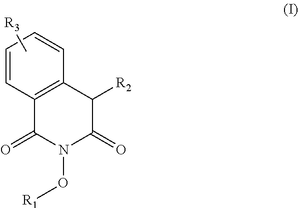
wherein:
R₁ is hydrogen or alkyl_{(C≤8)}, acyl_{(C≤8)}, or a substituted version of either of these groups;
R₂ is —C(O)ORₐ or —C(O)NRₐR_{b}, wherein:
Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
or a pharmaceutically acceptable salt thereof.

3. The method of claim 2, wherein the compound is further defined as: 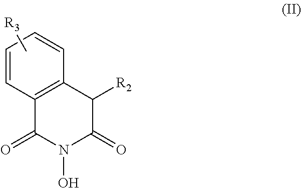
wherein:
R₂ is —C(O)ORₐ or —C(O)NRₐR_{b}, wherein:
Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
or a pharmaceutically acceptable salt thereof.

4. The method of claim 3, wherein R₂ is —C(O)ORₐ, wherein: Rₐ is hydrogen or alkyl_{(C≤8)}, aryl_{(C≤8)}, aralkyl_{(C≤8)}, or a substituted version of any of these groups.

5. The method of claim 4, wherein Rₐ is alkyl_{(C≤8)} or substituted alkyl_{(C≤8)}.

6. The method of claim 5, wherein Rₐ is methyl.

7. The method of claim 3, wherein R₂ is —C(O)NRₐR_{b}, wherein: Rₐ and R_{b} is hydrogen or alkyl_{(C≤8)}, aryl_{(C≤8)}, aralkyl_{(C≤8)}, or a substituted version of any of these groups.

8. The method of claim 7, wherein Rₐ is hydrogen.

9. The method of claim 7, wherein Rₐ is alkyl_{(C≤8)} or substituted alkyl_{(C≤8)}.

10. The method of claim 9, wherein Rₐ is ethyl.

11. The method of claim 7, wherein Rₐ is aryl_{(C≤8)} or substituted aryl_{(C≤8)}.

12. The method of claim 11, wherein Rₐ is phenyl.

13. The method of claim 7, wherein Rₐ is aralkyl_{(C≤8)} or substituted aralkyl_{(C≤8)}.

14. The method of claim 13, wherein Rₐ is benzyl, 4-fluorobenzyl, or 4-methoxybenzyl.

15. The method of claim 7, wherein R_{b} is hydrogen.

16. The method of claim 7, wherein R_{b} is alkyl_{(C≤8)} or substituted alkyl_{(C≤8)}.

17. The method of claim 16, wherein R_{b} is ethyl.

18. The method of claim 3, wherein R₃ is hydrogen.

19. The method of claim 3, wherein R₃ is halo.

20. The method of claim 19, wherein R₃ is chloro.

21. The method of claim 3, wherein R₃ is nitro.

22. The method of claim 3, wherein R₃ is alkoxy_{(C≤8)} or substituted alkoxy_{(C≤8)}.

23. The method of claim 22, wherein R₃ is methoxy.

24. The method of claim 3, wherein R₃ is acyl_{(C≤8)} or substituted acyl_{(C≤8)}.

25. The method of claim 22, wherein R₃ is —NHC(O)Ph.

26. The method according to any one of claims 1-3, wherein the compound is further defined as: 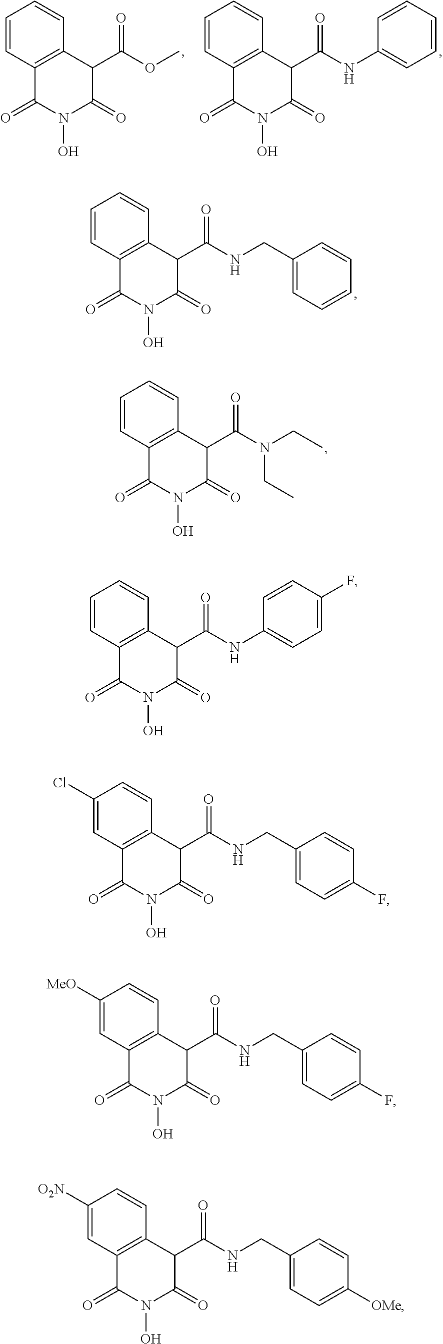 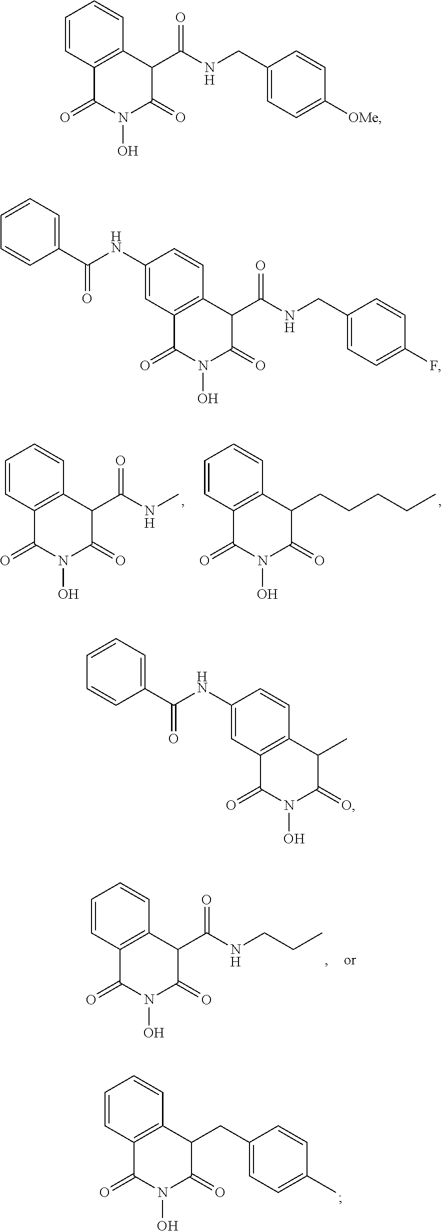
or a pharmaceutically acceptable salt thereof.

27. The method according to any one of claims 1-26, wherein the method is preformed in vivo.

28. The method according to any one of claims 1-26, wherein the method is preformed ex vivo.

29. The method according to any one of claims 1-26, wherein the method is preformed in vitro.

30. The method according to any one of claims 1-29, wherein the method is sufficient to treat an infection of a hepatitis B virus.

31. A method of treating a hepatitis B virus infection in a patient comprising administering to the patient in need thereof a pharmaceutically effective amount of a compound of the formula: 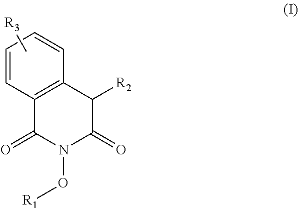
wherein:
R₁ is hydrogen or alkyl_{(C≤8)}, acyl_{(C≤8)}, or a substituted version of either of these groups;
R₂ is alkyl_{(C≤12)}, aryl_{(C≤12)}, aralkyl_{(C≤12)}, or a substituted version of any of these groups; or —C(O)ORₐ or —C(O)NRₐR_{b}, wherein:
Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
or a pharmaceutically acceptable salt thereof.

32. The method of claim 31, wherein the compound is further defined as: 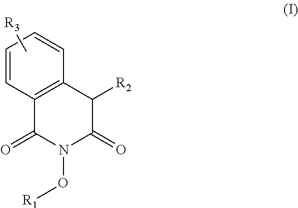
wherein:
R₁ is hydrogen or alkyl_{(C≤8)}, acyl_{(C≤8)}, or a substituted version of either of these groups;
R₂ is —C(O)ORₐ or —C(O)NRₐR_{b}, wherein:
Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
or a pharmaceutically acceptable salt thereof.

33. The method of claim 32, wherein the compound is further defined as: 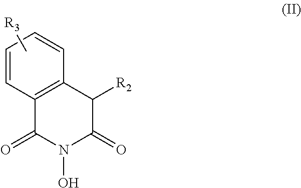
wherein:
R₂ is —C(O)ORₐ or —C(O)NRₐR_{b}, wherein:
Rₐ and R_{b} are each independently hydrogen or alkyl_{(C≤8)}, aryl_{(C≤8)}, aralkyl_{(C≤8)}, or a substituted version of any of these groups;
R₃ is hydrogen, amino, cyano, halo, hydroxy, or nitro, or alkyl_{(C≤8)}, cycloalkyl_{(C≤8)}, alkoxy_{(C≤8)}, or amido_{(C≤8)};
or a pharmaceutically acceptable salt thereof.

34. The method of claim 33, wherein R₂ is —C(O)ORₐ, wherein: Rₐ is hydrogen or alkyl_{(C≤8)}, aryl_{(C≤8)}, aralkyl_{(C≤8)}, or a substituted version of any of these groups.

35. The method of claim 34, wherein Rₐ is alkyl_{(C≤8)} or substituted alkyl_{(C≤8)}.

36. The method of claim 35, wherein Rₐ is methyl.

37. The method of claim 33, wherein R₂ is —C(O)NRₐR_{b}, wherein: Rₐ and R_{b} is hydrogen or alkyl_{(C≤8)}, aryl_{(C≤8)}, aralkyl_{(C≤8)}, or a substituted version of any of these groups.

38. The method of claim 37, wherein Rₐ is hydrogen.

39. The method of claim 37, wherein Rₐ is alkyl_{(C≤8)} or substituted alkyl_{(C≤8)}.

40. The method of claim 39, wherein Rₐ is ethyl.

41. The method of claim 37, wherein Rₐ is aryl_{(C≤8)} or substituted aryl_{(C≤8)}.

42. The method of claim 41, wherein Rₐ is phenyl.

43. The method of claim 37, wherein Rₐ is aralkyl_{(C≤8)} or substituted aralkyl_{(C≤8)}.

44. The method of claim 43, wherein Rₐ is benzyl, 4-fluorobenzyl, or 4-methoxybenzyl.

45. The method of claim 37, wherein R_{b} is hydrogen.

46. The method of claim 37, wherein R_{b} is alkyl_{(C≤8)} or substituted alkyl_{(C≤8)}.

47. The method of claim 46, wherein R_{b} is ethyl.

48. The method of claim 33, wherein R₃ is hydrogen.

49. The method of claim 33, wherein R₃ is halo.

50. The method of claim 49, wherein R₃ is chloro.

51. The method of claim 33, wherein R₃ is nitro.

52. The method of claim 33, wherein R₃ is alkoxy_{(C≤8)} or substituted alkoxy_{(C≤8)}.

53. The method of claim 52, wherein R₃ is methoxy.

54. The method of claim 33, wherein R₃ is acyl_{(C≤8)} or substituted acyl_{(C≤8)}.

55. The method of claim 52, wherein R₃ is —NHC(O)Ph.

56. The method according to any one of claims 31-33, wherein the compound is further defined as: 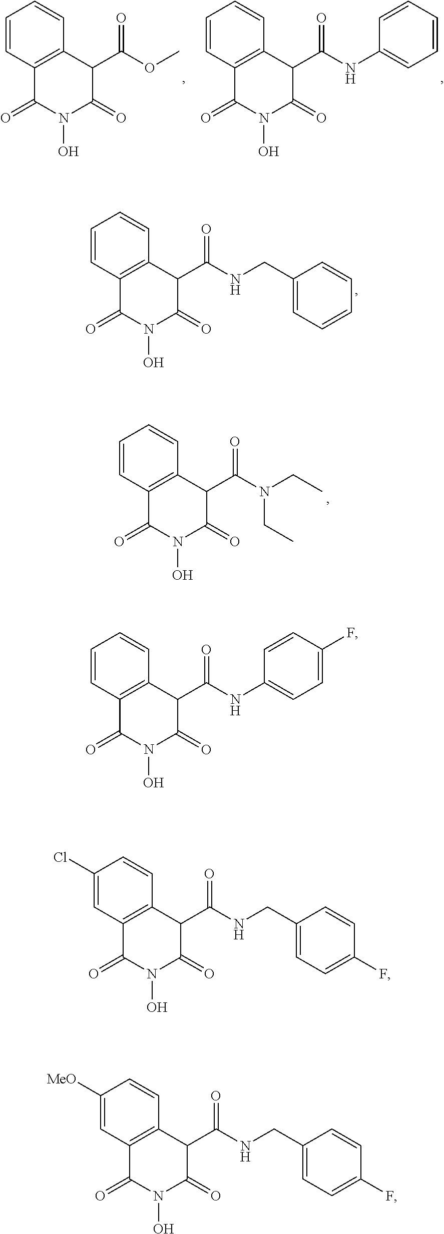 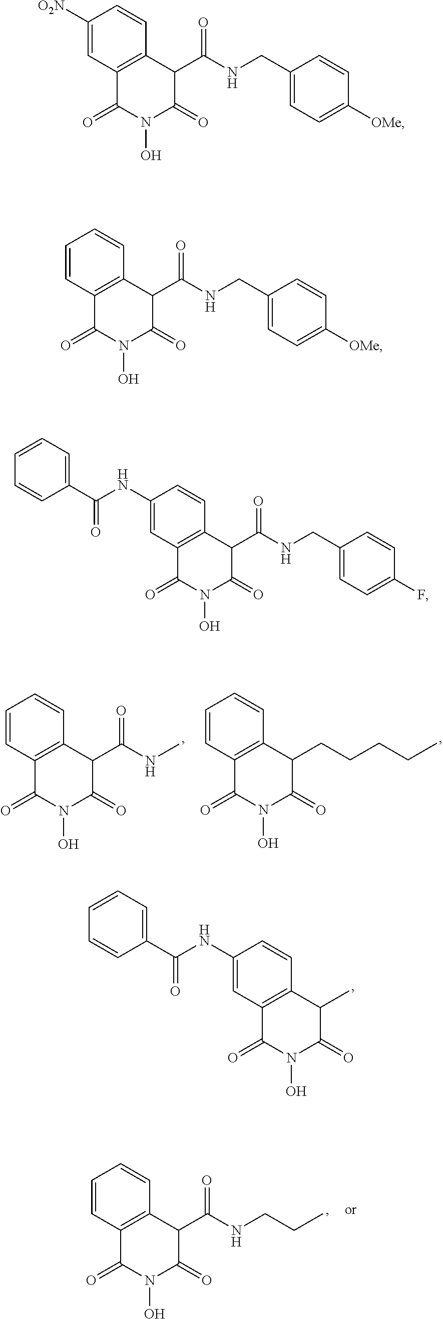 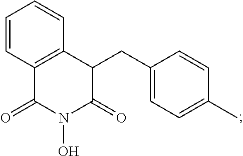 [SMILES: Cc1ccc(CC2C(=O)N(O)C(=O)c3ccccc32)cc1]
or a pharmaceutically acceptable salt thereof.

57. The method according to any one of claims 31-56, wherein the patient is a mammal.

58. The method of claim 57, wherein the patient is a human.

59. The method according to any one of claims 31-58, wherein the method further comprising a second antiviral therapy.

60. The method of claim 59, wherein the second antiviral therapy is interferon alfa-2b, lamivudine, adefovir, telbivudine, entercavir, or tenofovir.
